(19) United States
(12) Patent Application Publication
(10) Pub. No.: US 2021/0205443 A1
(43) Pub. Date: Jul. 8, 2021

(54) Carbohydrate-Modified Particles and Particulate Formulations for Modulating an Immune Response

(71) Applicant: NORTHWESTERN UNIVERSITY, Evanston, IL (US)

(72) Inventors: Paul J. Bryce, Chicago, IL (US); Karen B. Chien, Chicago, IL (US)

(21) Appl. No.: 17/247,045

(22) Filed: Nov. 24, 2020

Related U.S. Application Data
(63) Continuation of application No. 15/167,443, filed on May 27, 2016, now abandoned.
(60) Provisional application No. 62/167,054, filed on May 27, 2015.

(51) Int. Cl. A61K 39/385; A61K 39/00; A61K 39/35; A61K 47/69

(52) U.S. Cl. CPC A61K 39/385; A61K 39/0008; A61K 39/35; A61K 47/6937; A61K 2039/55555; A61K 2039/627; A61K 2039/572; A61K 2039/577; A61K 2039/6093

(57) ABSTRACT

Disclosed are compositions, kits, and methods for modulating an immune response. The compositions and kits include and the methods utilize carbohydrate-modified particles having an immune modulator attached at the surface of the particles. The carbohydrate-modified particles and particulate formulations comprising the carbohydrate-modified particles may be utilized for modulating an immune response in a subject.

### CROSS-REFERENCE TO RELATED PATENT APPLICATIONS

This Application is a Continuation of application Ser. No. 15/167,443 filed on May 27, 2016. Application Ser. No. 15/167,443 claims the benefit of U.S. Provisional Application 62/167,054 filed on May 27, 2015, the content of which is incorporated herein by reference in its entirety.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “40013A Seqlisting.txt”, which was created on Mar. 9, 2021 and is 2,237 bytes in size. The subject matter of the Sequence Listing is incorporated herein in its entirety by reference.

### BACKGROUND

The present invention relates generally to the field of compositions, kits, and methods for modulating an immune response. In particular, the invention relates to carbohydrate-modified particles and particulate formulations for modulating an immune response.

Methods for modulating immune responses are important for many disease treatments, and particle carriers have been examined for efficacy as delivery devices for proteins, drugs and other treatments. However, these carriers are largely innate themselves and typically function only as carriers for active components. Here, the inventors examined the possibility of functionalizing nanoparticle carriers, so as to actively modify the resulting immune response. Using an established nanoparticle material, poly(lactic-co-glycolic acid) or PLGA, and an in-house high-throughput screen for immunomodulatory co-signals, the inventors developed carbohydrate-enhanced nanoparticles (CENPs) that are capable of modulating immune responses.

### SUMMARY

Disclosed are compositions, kits, and methods for modulating an immune response. The compositions and kits include and the methods utilize carbohydrate-modified particles and particulate formulations comprising the carbohydrate-modified particles.

The carbohydrate-modified particles disclosed herein are relatively small and have an effective average diameter within a microscale or a nanoscale. Specifically, the carbohydrate-modified particles may be referred to as “carbohydrate-enhanced nanoparticles” or “CENPs.” The particles are modified via attachment of one or more carbohydrate moieties at the surface of the particles. Preferably, the particles are modified via covalent attachment of one or more carbohydrate moieties at the surface of the particles. The carbohydrate moieties may be attached directly to the surface of the particles or via one or more linker molecules. The carbohydrate moieties preferably function as immune modulators, for example, modulators that induce immune tolerance.

The disclosed particles of the compositions and formulations preferably are biodegradable and are formed from a polymeric base material. In some embodiments, the particles comprise polymeric base material formed from carbohydrate monomers or pre-polymers.

In addition to the carbohydrate moiety, the disclosed carbohydrate-modified particles may include additional components for modulating an immune response. In particular, the disclosed carbohydrate-modified particles may include an antigen, for example, a peptide, polypeptide, or protein that is utilized as an antigen and administered to a subject in order to desensitize the subject to the antigen and or to induce tolerance in the subject. Suitable antigens for inclusion in the disclosed carbohydrate-modified particle may include autoantigens associated with autoimmune disease (e.g., peptides, polypeptides, or proteins that are associated with autoimmune disease). Suitable antigens may include autoantigens associated with type 1 diabetes (T1D). Suitable antigens also may include antigens associated with allergic reactions (i.e., allergens).

The disclosed particles may be prepared by methods that include one or more of the following steps: (a) screening a library of carbohydrate moieties for immune-modulator activity by contacting the library with an immune cell and measuring the effect of the library on stimulating the immune cell (e.g., by measuring cytokine production over baseline and in particular IL-10, TGF(3, and/or CCL4 production versus IL-6 production); (b) selecting a carbohydrate moiety based on its effect on stimulating the immune cell; and (c) attaching the carbohydrate moiety thus selected to particles formed from a polymeric base material, preferably by covalently attaching the carbohydrate moiety to the surface of particles formed from a biodegradable polymeric base material.

The disclosed particles may be formulated as a composition for modulating an immune response. As such, the compositions may be administered to a subject in need thereof in order to induce an immune response, which may include but is not limited to desensitizing the subject and/or inducing tolerance in the subject. The compositions may be administered to treat and/or prevent diseases and disorders associated with autoimmune responses or to treat and/or prevent allergic reactions. The composition may be administered to treat and/or prevent transplant rejection.

### BRIEF DESCRIPTION OF THE DRAWING

FIG. 1 illustrates that in vitro stimulation (LPS) of macrophage by PLGA particles (PP) does not enhance IL-10 while EDC-cells (EDC SP) does enhance IL-10.

FIG. 2 illustrates a strategy for high throughput screening of carbohydrate compounds for induction of cytokine production by macrophage.

FIGS. 3A and 3B illustrate induction heat-maps for up-regulation or down-regulation of IL-10 response as determined using a high throughput screen as illustrated in FIG. 2.

FIGS. 4A-4B illustrates chemical coupling reactions for adding L-fucose to PLGA nanoparticles.

FIG. 5 illustrates that fucosylated PLGA (F-CENP) promote a stronger IL-10 induction than PLGA alone, EDC-cells, or free L-fucose.

FIG. 6 illustrates immunological mechanisms of sensitization and tolerance.

FIG. 7 illustrates potential therapies for treating allergies via desensitization and inducing tolerance.

FIG. 8 illustrates potential natural tolerogenic signals on the cell surface of an apoptotic cell.

FIG. 9 illustrates the hypothesis that the efficacy of an Ag-NP delivery system for tolerance therapy in T1D can be significantly enhanced by: (1) simultaneous engineering targeting ligands (LNFPIII and GAS6) on NPs for CD209 and Mer dual signaling; and (2) delivery of the deamidated form of insulin (INS (Q-*E)) as the initial disease-relevant autoantigen for inducing infectious tolerance.

FIGS. 10A, 10B, and 1C illustrate that AG-SP induces tolerance via expansion of Treg cells, AD deletion and anergy of Teff cells. FIG. 10A. CD4⁺Foxp3⁺ Treg cells in the spleen, dLN, and the graft in Ag-SP treated and control recipients on day 28 post transplantation. FIG. 10B. Congenically marked tha TCR transgenic T cells enumerated in the spleen, dLN, and the graft in Ag-SP treated and control recipients on day −4, day 0, and day 7. FIG. 10C. Congenically marked and CFSE labeled 4C TCR transgenic T cells examined for in vivo proliferation following first and second injection of Ag-SP. Histogram overlay also shows non-proliferating 4C T cells in untreated mice. (Kheradmand et al, J Immunol 189:804-12, 2012).

FIGS. 11A-11D. AG-SP injections induce expansion of MDSCs and soluble mediators implicated in Treg inducting and homing. FIG. 11A. Both Ly6C^{HI} and Gri^{m} MDSCs are expanded in numbers following Ag-SP injections. FIG. 11B. Co-culturing of Ly6C^{III} and Gr1ₘ MDSCs with stimulated T cells induces production of IL-10 and CCL4. FIG. 11C. Allografts from Ag-SP treated recipients show progressive accumulation of Foxp3⁺ Tregs. (Bryant et al, J Immunol 192(12): 6092, 2014).

FIGS. 12A, 12B, and 12C illustrate that Ag-SP-mediated MDSC expansion is dependent on the receptor tyrosine kinase MER. FIG. 12A. Two splenic macrophage populations expressing surface lectin CD209 and CD169 up-regulate Mer expression in response to Ag-SP treatment. FIG. 12B. Ag-SP induced expansion of Ly6C¹¹¹ and Gri^{m} MDSCs is lost in MerTK^{-/-} mice. FIG. 12C. Ag-SP tolerance therapy is ineffective in MerTIC^{/-} mice. This is in a BALB/c-*B6 heart transplant model, in which Ag-SP in MerTIC” (wild-type) mice significantly prolongs heart allograft survival, although not indefinite survival in contrast to that of islet allografts. (unpublished data).

FIGS. 13A, 13B, and 13C illustrate that NPs can be adapted for antigen delivery and tolerance induction. FIG. 13A. PLG NPs can be manufactured with specified size (in this case—500 nm) and zeta potential (in this case—75 mV). FIG. 13B. Donor antigens in the form of donor splenocyte lysate can be coupled to PLG NPs and safely delivered to recipient mice. However, the current form of Ag-NP provides only a marginal protection to the transplanted islet allograft when given alone. When combined with a short course of low dose rapamycin, the Ag- NP significantly improves its efficacy in islet allograft protection. (Bryant et al, Biomaterials 35: 8887-94, 2014).

FIGS. 14A, 14B, and 14C illustrate that humoral response to deamidated proinsulin in human T1D patients and in NOD mice. FIG. 14A. Antibody response to deamidated human proinsulin detected by Western blot in four of a cohort of 30 adult T1D patients. FIG. 14B. Top panel: representative antibody response to deamidated mouse proinsulin 1 by Western blot in a cohort of female NOD mice serially examined starting at 3 weeks of age. Bottom panel: diabetes incidence in subgroup female NOD mice with or without antibodies to deamidated proinsulin. FIG. 14C. 4×30 peptide array of murine proinsulin 1 and 2 probed by supernatant from positive NOD B cell hybridomas.

### DETAILED DESCRIPTION

Disclosed herein are compositions, kits, and methods for inducing an immune response against disease which may be described using several definitions as discussed below.

Unless otherwise specified or indicated by context, the terms “a,” “an,” and “the” mean “one or more.” In addition, singular nouns such as “carbohydrate” and “carbohydrate moiety” should be interpreted to mean “one or more carbohydrates” and “one or more carbohydrate moieties,” respectively, unless otherwise specified or indicated by context. Singular nouns such as “particle” should be interpreted to mean “one or more particles,” unless otherwise specified or indicated by context.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus <10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of should be interpreted as being “closed” transitional terms that do not permit the inclusion of additional components other than the components recited in the claims. The term “consisting essentially of should be interpreted to be partially closed and allowing the inclusion only of additional components that do not fundamentally alter the nature of the claimed subject matter.

The terms “subject,” “patient,” or “host” may be used interchangeably herein and may refer to human or non-human animals. Non-human animals may include, but are not limited to non-human primates, dogs, and cats.

The terms “subject,” “patient,” or “individual” may be used to refer to a human or non-human animal. A subject may include a human having or at risk for acquiring a disease and/or disorder that may be treated and/or prevented by immune-modulation, which may include desensitization and/or inducing tolerance. Diseases and/or disorders that are treated and/or prevented by immune-modulation may include but are not limited to allergies, including food allergies and other types of allergies. Diseases and/or disorders that are treated and/or prevented by immune-modulation may include autoimmune diseases and disorders such as autoimmune diseases of the heart (e.g., myocarditis and postmyocardial infarction syndrome), the kidney (e.g., anti-glomerular basement membrane nephritis), the liver (e.g., autoimmune hepatitis, primary biliary cirrhosis), the skin (e.g., alopecia areata, psoriasis, systemic scheroderma, and vitiligo), the adrenal gland (e.g., Addison's disease), the pancreas (e.g., autoimmune pancreatitis and diabetes mellitus type 1 (T1D)), the thyroid gland (e.g., Grave's disease), the salivary glands (e.g., Sjogren's syndrome), the digestive system (e.g., celiac disease, Crohn's disease, and ulcerative colitis), the blood (e.g., autoimmune thrombocytopenic purpura, Evans syndrome, pernicious anemia, and thrombocytopenia), the connective tissue (e.g., ankylosing spondylitis, juvenile arthritis, rheumatoid arthritis, sarcoidosis, and systemic lupus erythematosus), the muscle tissue (e.g., fibromyalgia, myasthenia gravis, and dermatomyositis), and the nervous system (e.g., acute disseminated encephalomyelitis, Guillain-Barre syndrome, multiple sclerosis, and idiopathic inflammatory demyelinating disease).

A subject may include a subject about to undergo a transplant operation or a subject that has undergone a transplant operation. A subject may include a subject about to undergo a transplant operation or a subject that has undergone a transplant operation where the subject is rejecting the transplant or is at risk for rejecting the transplant.

Disclosed herein are carbohydrate-modified particles. The carbohydrate-modified particles are relatively small and have an effective average diameter within a microscale or a nanoscale. In some embodiments, the carbohydrate-modified particles may have an effective average diameter of less than about 500 pm, 200 pm, 100 pm, 50 pm, 20 pm, 10 pm, 5 pm, 2 pm, 1 pm, 0.5 pm, 0.2 pm, 0.1 pm, 0.05 pm, 0.02 pm, 0.01 pm, or the carbohydrate-modified particles may have an effective average diameter within a range bounded by any of these values as endpoints such as 0.02-1 pm or 200-1000 nm. The carbohydrate-modified particles may be referred to herein as “microparticles” and/or “nanoparticles.” Specifically, the carbohydrate-modified particles may be referred to as “carbohydrate-enhanced nanoparticles” or “CENPs.”

The disclosed particles typically have a suitable zeta potential, for example, for administering the disclosed particles to a subject in need thereof. In some embodiments, the disclosed particles have a negative zeta potential, for example, within a range bounded by any of the following zeta potential values: −10 mV, −20 mV, −30 mV, −40 mV, −50 mV, −60 mV, −70 mV, −80 mV, −90 mV, or −100 mV, for example −50 to −100 mV or −60 to −80 mV.

The disclosed particles may comprise a biodegradable base material. The particles are “biodegradable” as would be understood in the art. The term “biodegradable” may be used to describe a material that is capable of being degraded in a physiological environment into smaller basic components. Preferably, the smaller basic components are innocuous. For example, an biodegradable polymer may be degraded into basic components that include, but are not limited to, water, carbon dioxide, sugars, organic acids (e.g., tricarboxylic or amino acids), and alcohols (e.g., glycerol or polyethylene glycol). Biodegradable materials that may be utilized to prepare the particles contemplated herein may include materials disclosed in U.S. Pat. Nos. 7,470,283; 7,390,333; 7,128,755; 7,094,260; 6,830,747; 6,709,452; 6,699,272; 6,527,801; 5,980,551; 5,788,979; 5,766,710; 5,670,161; and 5,443,458; and U.S. Published Application Nos. 20090319041; 20090299465; 20090232863; 20090192588; 20090182415; 20090182404; 20090171455; 20090149568; 20090117039; 20090110713; 20090105352; 20090082853; 20090081270; 20090004243; 20080249633; 20080243240; 20080233169; 20080233168; 20080220048; 20080154351; 20080152690; 20080119927; 20080103583; 20080091262; 20080071357; 20080069858; 20080051880; 20080008735; 20070298066; 20070288088; 20070287987; 20070281117; 20070275033; 20070264307; 20070237803; 20070224247; 20070224244; 20070224234; 20070219626; 20070203564; 20070196423; 20070141100; 20070129793; 20070129790; 20070123973; 20070106371; 20070050018; 20070043434; 20070043433; 20070014831; 20070005130; 20060287710; 20060286138; 20060264531; 20060198868; 20060193892; 20060147491; 20060051394; 20060018948; 20060009839; 20060002979; 20050283224; 20050278015; 20050267565; 20050232971; 20050177246; 20050169968; 20050019404; 20050010280; 20040260386; 20040230316; 20030153972; 20030153971; 20030144730; 20030118692; 20030109647; 20030105518; 20030105245; 20030097173; 20030045924; 20030027940; 20020183830; 20020143388; 20020082610; and 0020019661; the contents of which are incorporated herein by reference in their entireties. Typically, the particles disclosed herein are degraded in vivo at a degradation rate such that the particles lose greater than about 50%, 60%, 70%, 80%, 90%, 95%, or 99% of their initial mass after about 4, 5, 6, 7, or 8 weeks post-administration to a subject via one or more of: degradation of the biodegradable polymers of the particles to monomers: degradation of the biodegradable polymers of the particles to water, carbon dioxide, sugars, organic acids (e.g., tricarboxylic or amino acids), and alcohols (e.g., glycerol or polyethylene glycol); and degradation of the particles to release the carbohydrate-moiety of the particles or any immune modulatory agent present in the particles.

Suitable polymers for preparing the base material of the particles may include, but are not limited to, co-polymers of PLA and PGA (i.e., PLGA), mono-polymers such as polylactides (i.e., PLA) including polylactic acid, mono-polymers such as polyglycolides (i.e., PGA) including polyglycolic acid. Other suitable polymers may include, but are not limited to, polycaprolactone (PCL), poly(dioxanone) (PDO), collagen, renatured collagen, gelatin, renatured gelatin, cross-linked gelatin, and their co-polymers. The polymer of the particles is designed to degrade as a result of hydrolysis of polymer chains into biologically acceptable and progressively smaller components such as polylactides, polyglycolides, and their copolymers. These break down eventually into lactic and glycolic acid, enter the Kreb's cycle and are broken down into carbon dioxide and water and excreted.

In addition to the carbohydrate moiety, the disclosed carbohydrate-modified particles may include additional components for modulating an immune response. In particular, the disclosed carbohydrate-modified particles may include an antigen, for example, an antigen utilized and administered to a subject in order to desensitize the subject to the antigen and or to induce tolerance in the subject. The antigen may be covalently or otherwise attached to the surface of the carbohydrate-modified particles. Suitable antigens also may include antigens associated with allergic reactions, for example antigens associated with food allergies. Suitable antigens for inclusion in the disclosed carbohydrate-modified particle may include autoantigens associated with autoimmune disease, such as antigens associate with autoimmune diseases selected from, but not limited to autoimmune diseases of the heart (e.g., myocarditis and postmyocardial infarction syndrome), the kidney (e.g., anti-glomerular basement membrane nephritis), the liver (e.g., autoimmune hepatitis, primary biliary cirrhosis), the skin (e.g., alopecia areata, psoriasis, systemic scheroderma, and vitiligo), the adrenal gland (e.g., Addison's disease), the pancreas (e.g., autoimmune pancreatitis and diabetes mellitus type 1 (T1D)), the thyroid gland (e.g., Grave's disease), the salivary glands (e.g., Sjogren's syndrome), the digestive system (e.g., celiac disease, Crohn's disease, and ulcerative colitis), the blood (e.g., autoimmune thrombocytopenic purpura, Evans syndrome, pernicious anemia, and thrombocytopenia), the connective tissue (e.g., ankylosing spondylitis, juvenile arthritis, rheumatoid arthritis, sarcoidosis, and systemic lupus erythematosus), the muscle tissue (e.g., fibromyalgia, myasthenia gravis, and dermatomyositis), and the nervous system (e.g., acute disseminated encephalomyelitis, Guillain-Barre syndrome, multiple sclerosis, and idiopathic inflammatory demyelinating disease).

In some embodiments of the disclosed carbohydrate-modified particles, in addition to the carbohydrate moiety, the disclosed carbohydrate-modified particles may include an antigen or allergen, for example, where the carbohydrate-modified particles may be administered to a subject exhibiting an allergic reaction to the antigen or allergen or at risk for developing an allergic reaction to the antigen or allergen in order to desensitize the subject to the antigen or allergen and/or to induce tolerance in the subject to the antigen or allergen. In other embodiments of the disclosed carbohydrate-modified particles, in addition to the carbohydrate moiety, the disclosed carbohydrate-modified particles may include an antigen derived from insulin, for example, where the carbohydrate-modified particles may be administered to a subject having type 1 diabetes or at risk for developing type 1 diabetes in order to desensitize the subject to insulin and/or to induce tolerance in the subject to insulin. In further embodiments of the disclosed carbohydrate-modified particles, in addition to the carbohydrate moiety, the disclosed carbohydrate-modified particles may include an antigen derived from a transplant in order to desensitize the subject to the antigen of the transplant and/or to induce tolerance in the subject to the antigen of the transplant and treat and/or prevent rejection of the transplant.

Suitable antigens for inclusion in the carbohydrate-modified particles, may include peptides, polypeptides, or proteins. As used herein, the terms “peptide,” “polypeptide,” and “protein,” which may be referred to herein interchangeable, refer to molecules that comprises polymers of amino acids. Where “amino acid sequence” is recited to refer to a sequence of a naturally occurring protein molecule, “amino acid sequence” and like terms are not meant to limit the amino acid sequence to the complete native amino acid sequence associated with the recited protein molecule. The term “amino acid” may refer to naturally occurring and/or non-naturally occurring amino acids.

As contemplated herein, peptides, polypeptides, and proteins may be utilized as antigens, for example, antigens that are covalently attached to the surface of the particles disclosed herein. For example, SEQ ID NOs:1-9 provide amino acid sequences of portions of insulin or variants thereof (e.g., Q-*E deamidated variants), which may be utilized as antigens as contemplated herein. Exemplary peptides, polypeptides, and proteins may comprise the amino acid sequence of any of SEQ ID NOs:1-9, or may comprises an amino acid sequence having at least about 80%, 90%, 95%, 96%, 97%, 98%, or 99% sequence identity to any of SEQ ID NOs:1-9. Variant peptides, polypeptides, and proteins may include polypeptides having one or more amino acid substitutions, deletions, additions and/or amino acid insertions relative to a reference peptides, polypeptides, and proteins.

The amino acid sequences contemplated herein may include conservative amino acid substitutions relative to a reference amino acid sequence. For example, a variant insulin polypeptide may include conservative amino acid substitutions relative to the natural insulin polypeptide. “Conservative amino acid substitutions” are those substitutions that are predicted to interfere least with the properties of the reference polypeptide. In other words, conservative amino acid substitutions substantially conserve the structure and the function of the reference protein. The following table provides a list of exemplary conservative amino acid substitutions.

| Original | Conservative |
|---|---|
| Residue | Substitution |
| Ala | Gly, Ser |
| Arg | His, Lys |
| Asn | Asp, Gln, His |
| Asp | Asa, Glu |
| Cys | Ala, Ser |
| Gin | Asn, Glu, His |
| Glu | Asp, Gln, His |
| Gly | Ala |
| His | Asn, Arg, Gln, flu |
| Ile | Leu, Val |
| Lcu | Lk, Val |
| Lys | Arg, Gln, Glu |
| Met | Leu, Ile |
| Phe | His, Met, Leu, Tip, Tyr |
| Ser | Cys, Thr |
| Thr | Ser, Wl |
| Trp | Phe, Tyr |
| Tyr | His, Phe, Trp |
| Wl | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides relative to a reference sequence. A deletion removes at least 1, 2, 3, 4, 5, 10, 20, 50, 100, or 200 amino acids residues or nucleotides. A deletion may include an internal deletion or a terminal deletion (e.g., an N-terminal truncation or a C-terminal truncation of a reference polypeptide or a 5′-terminal or 3′-terminal truncation of a reference polynucleotide).

A “fragment” is a portion of an amino acid sequence or a polynucleotide which is identical in sequence to but shorter in length than a reference sequence. A fragment may comprise up to the entire length of the reference sequence, minus at least one nucleotide/amino acid residue. For example, a fragment may comprise from 5 to 1000 contiguous nucleotides or contiguous amino acid residues of a reference polynucleotide or reference polypeptide, respectively. In some embodiments, a fragment may comprise at least 5, 10, 15, 20, 25, 30, 40, 50, 60, 70, 80, 90, 100, 150, 250, or 500 contiguous nucleotides or contiguous amino acid residues of a reference polynucleotide or reference polypeptide, respectively. Fragments may be preferentially selected from certain regions of a molecule. The term “at least a fragment” encompasses the full length polynucleotide or full length polypeptide.

“Homology” refers to sequence similarity or, interchangeably, sequence identity, between two or more polynucleotide sequences or two or more polypeptide sequences. Homology, sequence similarity, and percentage sequence identity may be determined using methods in the art and described herein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

A “variant” of a particular polypeptide sequence is defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of polypeptides may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides.

The disclosed polypeptides may be modified so as to comprise an amino acid sequence or modified amino acids, such that the disclosed polypeptides cannot be said to be naturally occurring. In some embodiments, the disclosed polypeptides are modified and the modification is selected from the group consisting of acylation, acetylation, formylation, lipolylation, myristoylation, palmitoylation, alkylation, isoprenylation, prenylation, and amidation. An amino acid in the disclosed polypeptides may be thusly modified, but in particular, the modifications may be present at the N-terminus and/or C-terminus of the polypeptides (e.g., N-terminal acylation or acetylation, and/or C-terminal amidation). The modifications may enhance the stability of the polypeptides and/or make the polypeptides resistant to proteolysis.

The disclosed particles may be prepared by methods known in the art including, but not limited to, methods disclosed in U.S. Pat. Nos. 8,546,371; 8,518,450; and 7,550,154, the contents of which are incorporate herein by reference in their entireties. Methods for forming microparticles and/or nanoparticles may include, but are not limited to spray-drying, precipitation, and/or grinding a base material (e.g., a biodegradable, polymeric base material).

The disclosed particles typically are modified via inclusion of a carbohydrate moiety, preferably a carbohydrate moiety that is an immune modulator attached at the surface of the particles (e.g., via covalent attachment). Suitable carbohydrate moieties may include, but are not limited to moieties from the following group or pharmaceutical salts thereof: Heparin disaccharide I-A, Heparin disaccharide II-A, Heparin disaccharide III-A, Heparin disaccharide IV-A, Heparin disaccharide IV-S, Heparin unsaturated disaccharide I-H, Heparin unsaturated disaccharide II-H, Heparin unsaturated disaccharide II-H, Heparin unsaturated disaccharide I-P, Chondroitin disaccharide Adi-OS, Chondroitin disaccharide Adi-45, Chondroitin disaccharide Adi-65, Chondroitin disaccharide ADi-diSB, Chondroitin disaccharide ADi-diSE, Chondroitin disaccharide ADi-tris, Chondroitin disaccharide ADi-UA2S, Neocarradecaose-41,3,5,7,9-penta-0-sulphate, neocarrahexadecaose-41,3,5,7,9,11,13,15-octa-0-sulfate, GalNAc131-4Gal (receptor for pili of Pseudomonas aeruginosa), Blood group B type 2 linear trisaccharide, P1 Antigen, Tn Antigen, Sialyl-Lewis A, Sialyl-Lewis X, Sialyl-Lewis X 13-methyl glycoside, Sulfo-Lewis A, Sulfo-Lewis X, al-2-Mannobiose, al-3-Mannobiose, al-6-Mannobiose, Mannotetraose, al-3, al-3, al-6-Mannopentose, 131-2-N-Acetylglucosamine-mannose, LS-Tetrasaccharide a (LSTa), LS-tetrasaccharide c (LSTc), a-D-N-Acetylgalactosaminyl 1-3 galactose, a-D-N-Acetylgalactosaminyl 1-3 galactose 131-4 glucose, D-Galactose-4-0-sulfate, Glycyl-lactose (Lac-gly), Glycyl-lacto-N-tetraose (LNT-gly), 2′-Fucosyllactose, Lacto-N-neotetraose (LNnT), Lacto-N-tetraose (LNT), Lacto-N-difucohexaose I (LNDFH I), Lacto-N-difucohexaose II (LNDFHII), Lacto-N-neohexaose (LNnH), 3′-Sialyllactose (3′-SL), 6′-Sialyllactose (6′-SL), 3′-Sialyl-N-acetyllactosamine, 6′-Sialyl-N-acetyllactosamine (6′-SLN), 3-Fucosyllactose (3FL), Fucoidan, 4-13-Galactobiose, 1-3 Galactodiosyl 13-methyl glycosie, al-3, 131-4, al-3 Galactotetraose, 13-Galactosyl 1-3 N-acetyl galactosamine methyl glycoside, 131-3 Gal-N-acetyl galactosaminyl-131-4 Gal-131-4-Glc, 131-6 Galactobiose, Globotriose, 13-D-N-Acetylglactosaminyl 1-3 galactose (terminal disaccharide of globotriose), 1-Deoxynojirimyncin (DNJ), D-Fucose, L-Fucose, D-Talose, Calystegine A3, Calystegine B3, N-methyl cis-4-hydroxymehtyl-L-proline, 2,5-dideoxy-2,5-imino-D-mannitol, Castanospermine, 6-epi-Castanospermine, and combinations thereof. In some embodiments, the particles comprise multiple carbohydrate moieties and are tailored to treat and/or prevent a disease or disorder via immune-modulation.

The carbohydrate moieties of the disclosed particles typically are carbohydrates consisting of carbon, hydrogen, and oxygen atoms and may have an empirical formula Cₘ(1-1₂0), where m and n are integers and may be the same or different. Some carbohydrates may include atoms other than carbon, hydrogen, and oxygen, for example, nitrogen, phosphorus, and/or sulfur atoms. However, carbohydrates that include atoms other than carbon, hydrogen, and oxygen, for example, nitrogen, phosphorus, and/or sulfur atoms, typically include these other atoms at a small molar mass fraction of the carbohydrate molecule (e.g., less than 10% or 5%).

The carbohydrate moieties may be attached directly to the surface of the particles (e.g., via covalent coupling). Optionally, the carbohydrate moieties may be attached indirectly to the surface of the particles, for example, covalently via one or more linker molecules (e.g., a polyethylene glycol linker). The carbohydrate moieties may be attached to the surface of the particles via crosslinking methods that may include but are not limited to carbodiimide (EDC) crosslinking.

Optionally, the disclosed particles may comprise one or more additional immunomodulatory agents other than the carbohydrate moiety. Additional agents may include antigens as discussed above, and/or cytokines (e.g., interleukins and interferons) and/or immune-modulatory antibodies.

The disclosed particles function as “immuno-enhancers” and/or “immuno-inhibitors.” As such, the disclosed particles may be administered in a number of applications, including but not limited to—immunoenhancing to improve vaccine efficacy; immunoenhancing to improve anti-tumor immunity and cancer outcomes; immunoenhancing to improve outcomes during infectious disease; immunoinhibiting to treat allergic diseases, such as asthma, food allergy and eczema; immunoinhibiting to treat autoimmune diseases, such as rheumatoid arthritis, multiple sclerosis and diabetes; and/or immunoinhibiting to improve outcomes during transplantation.

The disclosed particles may be administered in order to desensitize a subject and/or induce tolerance in the subject to an antigen. Desensitization and/or tolerance may be assessed using methods in the art and disclosed herein which may include, but are not limited to preferably inducing secretion of IL-10, TGF13, or CCL4 by macrophages over baseline versus inducing secretion of IL-6 over baseline. As such, desensitization and/or tolerance may be assessed using a ratio IL-10/IL-6 which reflects the relative change in secretion of IL-10 over baseline versus the change in secretion of IL-6 over baseline.

The disclosed particles may be administered in order to modulate an immune response in a subject. As such, the disclosed particles may be formulated as a pharmaceutical composition. Such compositions can be formulated and/or administered in dosages and by techniques well known to those skilled in the medical arts taking into consideration such factors as the age, sex, weight, and condition of the particular patient, and the route of administration.

The compositions may include pharmaceutical solutions comprising carriers, diluents, excipients (e.g., powder excipients such as lactose, sucrose, and mannitol), and surfactants (e.g., non-ionic surfactants), as known in the art. Further, the compositions may include preservatives (e.g., anti-microbial or anti-bacterial agents). The compositions also may include buffering agents (e.g., in order to maintain the pH of the composition between 6.5 and 7.5).

The pharmaceutical compositions may be administered prophylactically or therapeutically. In prophylactic administration, the composition may be administered to a subject in an amount sufficient to modulate an immune response for protecting against a disease or disorder (i.e., a “prophylactically effective dose”)). In therapeutic applications, the compositions are administered to a subject in an amount sufficient to treat a disease or disorder (i.e., a “therapeutically effective dose”)).

The compositions disclosed herein may be delivered via a variety of routes. Typical delivery routes include parenteral administration (e.g., intradermal, intramuscular, intraperitoneal, or subcutaneous delivery). Other routes include intranasal and intrapulmonary routes. Formulations of the pharmaceutical compositions may include liquids (e.g., solutions and emulsions), sprays, and aerosols. In particular, the compositions may be formulated as aerosols or sprays for intranasal or intrapulmonary delivery. Suitable devices for administering aerosols or sprays for intranasal or intrapulmonary delivery may include inhalers and nebulizers.

The compositions disclosed herein may be co-administered or sequentially administered with other immunological, antigenic or vaccine or therapeutic compositions, including an adjuvant, or a chemical or biological agent given in combination with an antigen to enhance immunogenicity of the antigen. Additional therapeutic agents may include, but are not limited to, cytokines such and interleukins and interferons.

As used herein, a “prime-boost vaccination regimen” refers to a regimen in which a subject is administered a first composition and then after a determined period of time (e.g., after about 2, 3, 4, 5, or 6 weeks), the subject is administered a second composition, which may be the same or different than the first composition. The first composition (and the second composition) may be administered one or more times. The disclosed methods may include priming a subject with a first composition by administering the first composition at least one time, allowing a predetermined length of time to pass (e.g., at least about 2, 3, 4, 5, or 6 weeks), and then boosting by administering the same composition or a second, different composition.

In order to assess the efficacy of the pharmaceutical compositions disclosed herein, an immune response can be assessed by measuring the induction of cell-mediated responses and/or antibody responses. T-cell responses may be measured, for example, by using tetramer staining of fresh or cultured PBMC, ELISPOT assays or by using functional cytotoxicity assays, which are well-known to those of skill in the art. Antibody responses may be measured by assays known in the art such as ELISA. Titer or load of a pathogen may be measured using methods in the art including methods that detect nucleic acid of the pathogen. (See, e.g., U.S. Pat. No. 7,252,937, the content of which is incorporated by reference in its entirety).

### EXAMPLES

The following examples are illustrative and are not intended to limit the disclosed subject matter.

### Example 1

### Carbohydrate Enhanced Nanoparticles for Immune Modulation

Introduction

PLGA nanoparticles have been utilized for a variety of applications, including drug delivery, tissue and cellular imaging, and for delivering self or foreign proteins to aid induction of immune activation or tolerance. (See Sah et al., “Concepts and practices used to develop functional PLGA-based nanoparticulate systems,” International Journal of Medicine, 2013:8 747-765). Here, we have developed the technology for generating functionalized PLGA particles and have established their potential for modifying the immune response.

Experimental Methods, Results, and Discussion

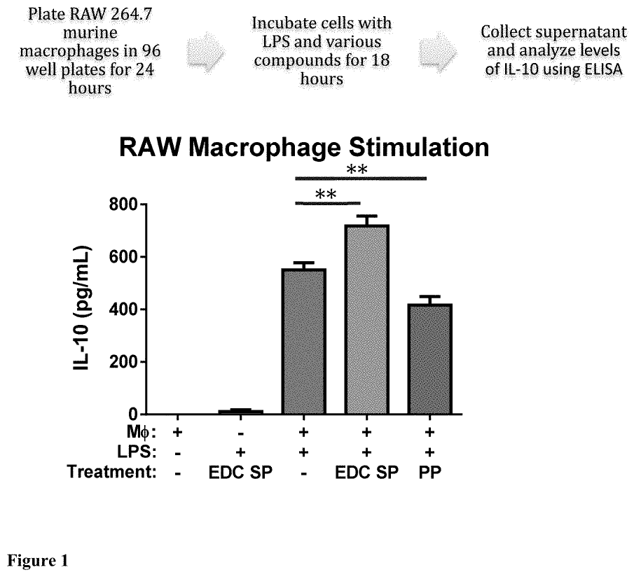

We initially approached this area from our research on cell-coupled antigen tolerance, as a therapeutic approach to treating allergic disease (see Smarr et al., “Antigen-fixed leukocytes tolerize Th2 responses in mouse models of allergy,” The Journal of Immunology, 11/2011; 187(1):5090-8), whereby allergic proteins are attached to autologous cells using EDC carbodiimide crosslinking chemistry, infused back into mice and tolerance (i.e., a state of immunological unresponsiveness) is generated. Since applying this to patients is complicated by the use of cells, we began examining the potential use of PLGA nanoparticles as replacements; however our findings showed antigen encapsulated within PLGA nanoparticles induced a different response (desensitization, rather than tolerance, and so reactivity recovered after time). Since macrophages are one immune cell thought to be important in immune responses, including tolerance through their production of a key immune mediator called Interleukin-10 (IL-10), we developed an in vitro approach to screening the effects of EDC-cells versus PLGA nanoparticles for their effects on IL-10 production. As shown in FIG. 1, we observed that EDC-cells enhanced IL-10 after stimulation (Lipopolysaccaride (LPS)), while PLGA particles did not.

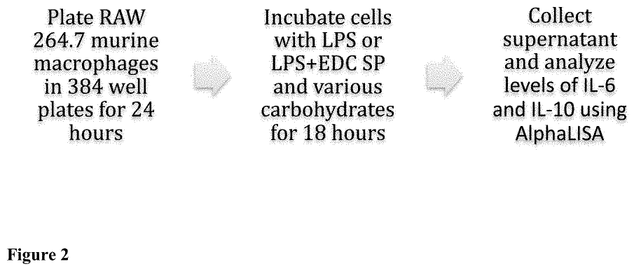

Based on this, we concluded that signals present on the cells were not present on the PLGA particles and so developed a High Throughput-based screen (Performed through the Northwestern HTS core in Evanston). We examined the proinflammatory response (IL-6) or anti-inflammatory response (IL-10), as outlined in FIG. 2.

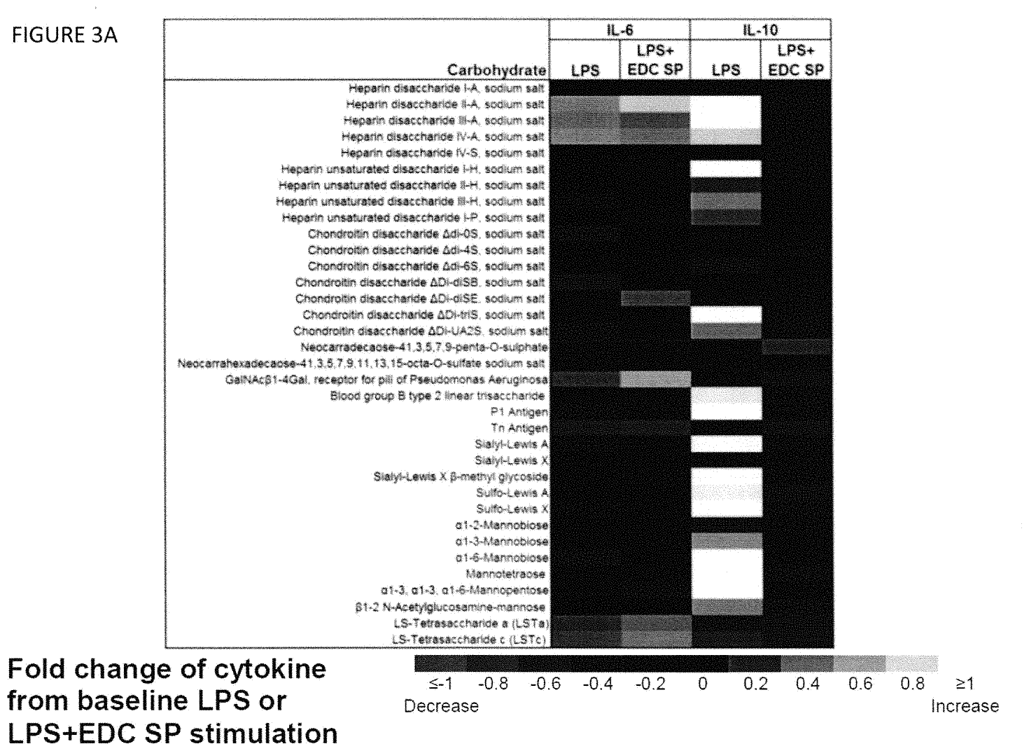

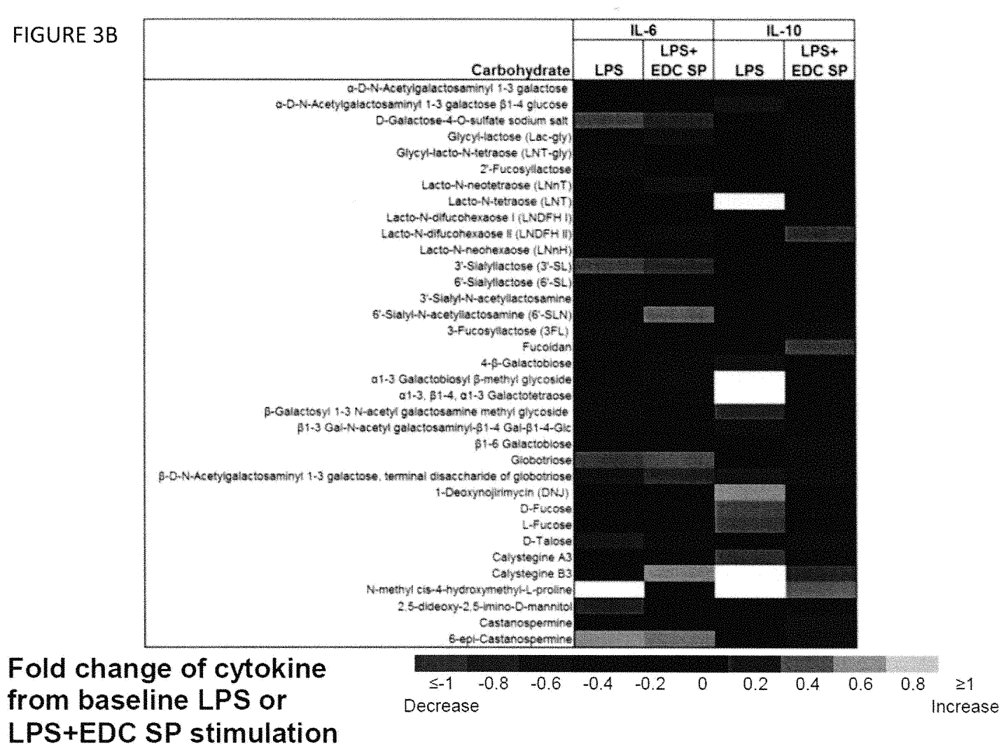

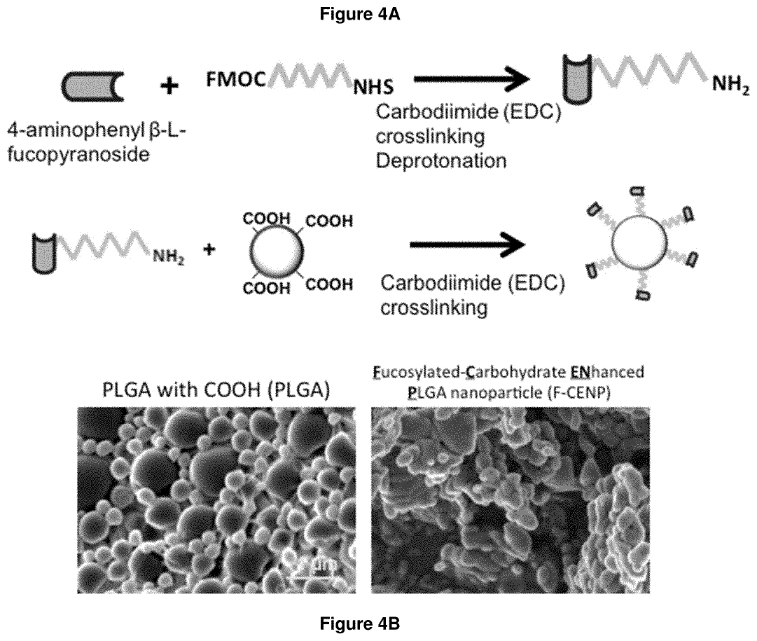

We examined a panel of 70 unique carbohydrate compounds that are found on cells but would not be present on PLGA nanoparticles. Using the strategy in FIG. 2, we calculated the greatest fold change for cytokine production over a therapeutically relevant dose curve of 0.1, 1, 10, and 100 μM. As represented in FIG. 3, we identified many carbohydrate compounds that were capable of modifying the macrophage response, with both upregulation, downregulation, or no change compared to EDC-cells or stimulation alone, with the suggestion that these had potential to functionalize PLGA if coupled to the particles. To pursue this further, we chose one candidate (L-fucose) and coupled it to PLGA using a 2-stage chemical process. The coupling process is shown in FIG. 4.

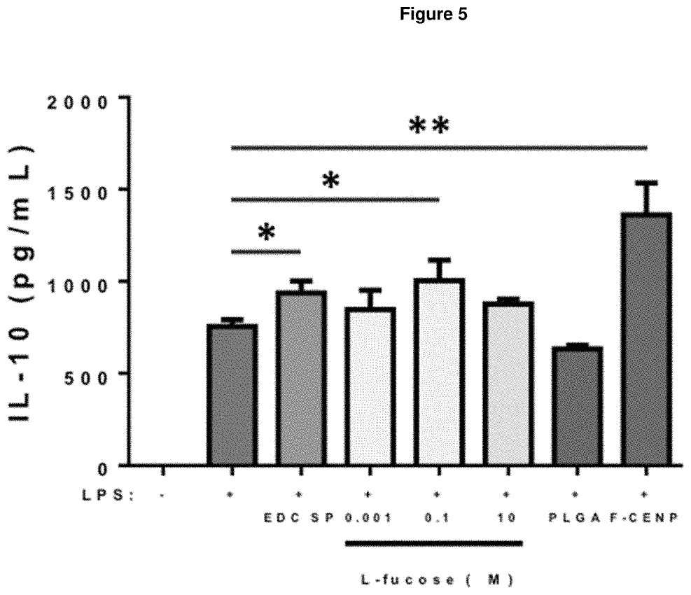

Initially, a derivative of L-fucose (4-aminophenyl beta-L-fucopyranoside) was attached to a poly(ethylene glycol) (PEG) linker using an EDC crosslinking reaction. This was then attached to carboxylated PLGA nanoparticles using a second EDC crosslinking reaction. Characterization of the end product showed loss of the spherical structure of uncoupled particles and a rough, irregular particle. To test the functional abilities of the fucosylated PLGA nanoparticles (termed F-CENP), we examined their effects on our in vitro model for IL-10 production. As shown in FIG. 5, F-CENP was significantly better at IL-10 induction than cells receiving PLGA alone, L-fucose alone or even EDC-cells, suggesting the functionalized PLGA particle is an improvement over even EDC-cells.

### Example 2

### Development of Carbohydrate Enhanced Nanoparticles for the Induction of Immune Tolerance in Food Allergies

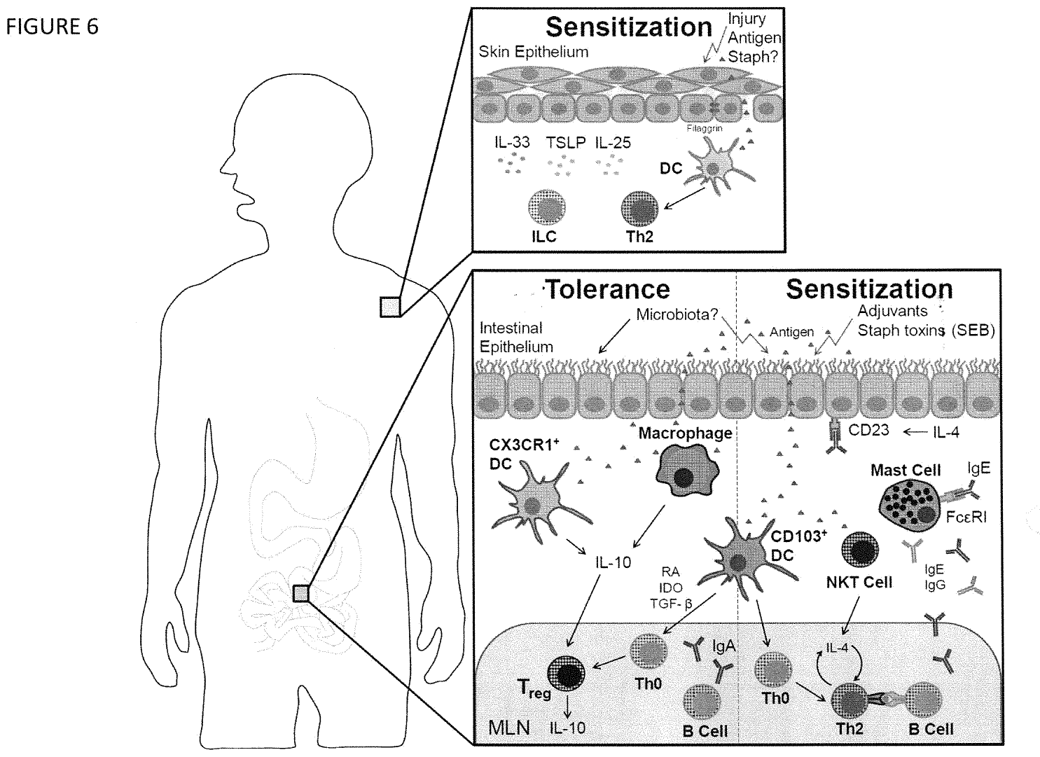

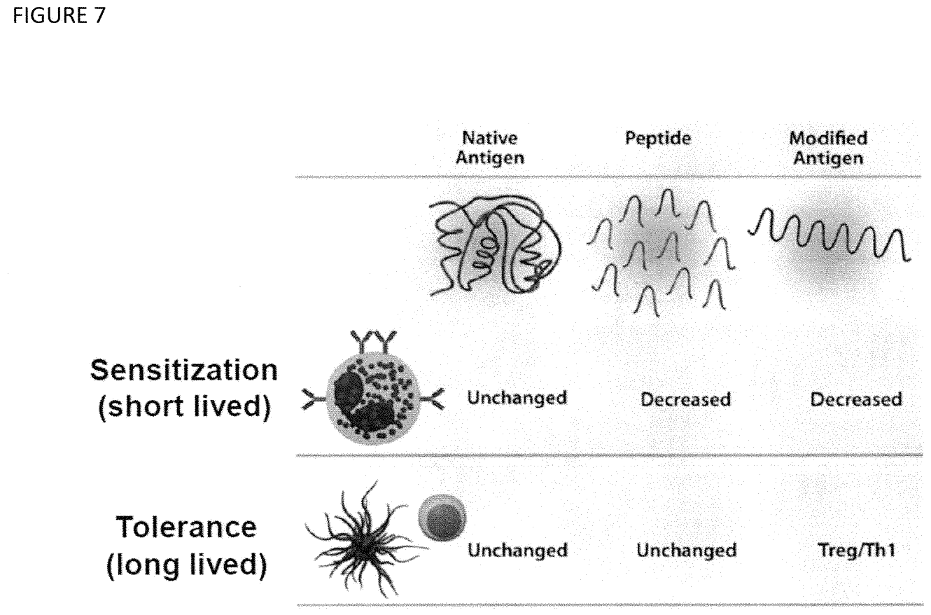

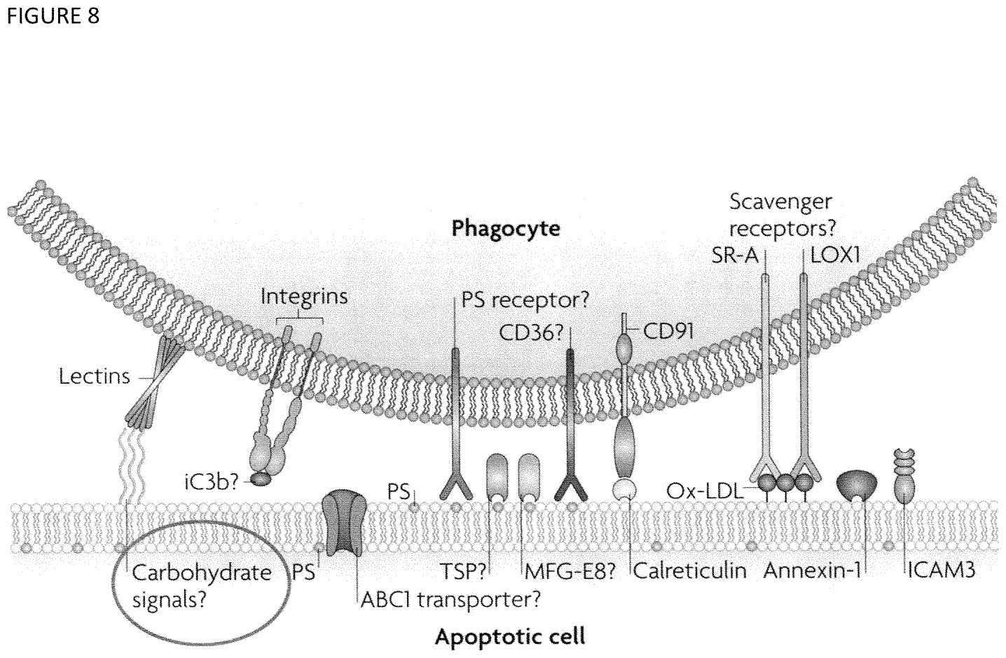

Food allergies may be defined as an adverse immune reaction to foods and may include hives and life threatening anaphylaxis. The severity of the reaction can depend on a number of factors including the amount of food ingested, the form of the food (e.g., raw, cooked, or processed), and risk factors such as age, degree of sensitization, and other comorbid conditions. Food allergies are classically known as being IgE-mediated but can be heterogeneous in physiological responses and symptoms. (See Sicherer and Sampson, J Allergy Clin. Immunol. (2010) February; 125(2 Suppl 2)S116-25; Berin and Mayer, J. Allergy Clin. Immunol. (2013) January; 131(1):14-22; and Boyce et al. (NIAID Guidelines), J Allergy Clin. Immunol. (2010) December; 126(6 Suppl):S1-58). The immunological mechanisms involved in food allergies include sensitization and tolerance, (see Johnston et al., J Immunol. (2014) March 15; 192(6):2529-34, and FIG. 6), and potential therapies for food allergies may involve administering antigen for desensitization (short lived therapy) and/or for increasing tolerance (long lived therapy) (Berin and Mayer, J Allergy Clin. Immunol. (2013) Jan;131(1):14-22, and FIG. 7). Antigens encapsulated in microparticle have been administered in a food allergy model in order to induce desensitization, and antigen-fixed leukocytes have been shown to tolerize responses in mouse models of allergy. (See Smarr et al., J Immunol. (2011) 187:5090-5098). However, an ideal engineered therapeutic should provide not only antigen to induce desensitization or tolerance, but also concurrent tolerogenic signals to the immune system. As such, methods for identifying tolerogenic signals that may be utilized allergy therapy involving desensitization and tolerance are desirable. Once identified, the tolerogenic signals may be formulated as part of micro-and/or nano-particles which optionally include antigens for inducing desensitization and/or tolerance. Apoptotic cells include natural tolerogenic signals on the cell surface. (See Taylor et al., Nat. Rev. Mol. Bio. (2008) March; 9(3):231-41, and FIG. 8). Compounds present on the cell surface including proteins, lipids, glycolipids, and carbohydrates, which may be involved in the development of tolerance.

Allergic responses generally involve an inflammatory response, and LPS-stimulated macrophages (i.e. “activated macrophages”) have been used as a tool for studying skewing of the inflammatory response. For example, LPS-stimulated macrophages secrete pro-inflammatory cytokines such as IL-6, TNF-α, and 11113, and modulation of the secretion of these inflammatory cytokines can be used to identify compounds that inhibit the inflammatory response. Chemical compounds that have been found to inhibit this inflammatory response in RAW 264.7 macrophage, characterized by a decrease in secretion of the pro-inflammatory cytokines and an increase in IL-10/TGF13, include: 6-dehydrogingerdione; peimine; adenosine; and saikosaponin A. (See Huang et al., J. Agric. Food Chem. (2014) Seep 17; 62(37):9171-9; Yi et al., Immunopharmacol. Immunotoxicol. (2013) October; 35(5):567-72; au et al., Exp. Ther. Med. (2013) May;5(5):1345-1350; and Koscso et at, J. Leukoc. Biol. (2013) December; 94(6):1309-15). Accordingly, activated RAW macrophages may be used as a model to screen for tolerogenic signals.

Using activated RAW macrophages we developed a high throughput screening method to identify tolerogenic signals. (See FIG. 2). We tested seventy (70) compounds based on a compound's ability to increase IL-10 secretion versus baseline and/or to decrease IL-6 secretion versus baseline in macrophages activated with LPS and in macrophages activated with LPS in the presence of splenocytes (SP) that have been treated with the chemical cross-linker ethylcarbodiimide (ECDI-SP). Antigens that are crosslinked to the surface of ECDI-SP may be administered in induce antigen-specific tolerance (see Jenkins et al., J. Exp. Med. 165:302-319 (1987)), and as such, we included macrophages activated with LPS in the presence of ECDI-SP to determine whether a compound would exhibit similar tolerogenic signals in macrophages activated with LPS and in macrophages activated with LPS in the presence of ECDI-SP. We identified a number of carbohydrate compounds that exhibit tolerogenic signals. (See FIG. 3A and 3B). Fucose was selected as an exemplary carbohydrate compounds exhibiting a tolerogenic signal and was coupled to nanoparticles having a PLGA polymer core to generate carbohydrate-enhanced PLGA nanoparticles (F-CENP). (See FIG. 4). As shown in FIG. 5, F-CENP was significantly better at IL-10 induction than cells receiving PLGA alone, L-fucose alone or even EDC-cells, suggesting the functionalized PLGA particle is an improvement over even EDC-cells.

In summary, RAW macrophages can be used as a screening system to identify potential compounds that may induce tolerance. Our preliminary screening of 70 compounds revealed several compounds that could be used to promote IL-10 secretion while not changing or decreasing IL-6 secretion. Our results indicate that tolerance-promoting signals may be incorporated into a therapeutic design for administering antigen and inducing tolerance with greater efficiency.

### Example 3

### LNFPIII and GAS6 Signaling Nanoparticles for Tolerance Delivery in T1D

Background

Type 1 diabetes (T1D) is an autoimmune disorder caused by autoreactive T cell-mediated destruction of the pancreatic 13 cells, resulting in hyperglycemia requiring exogenous insulin therapy. Individuals with a high risk for developing T1D can now be identified with a combination of genotyping for human leukocyte antigens and serological testing for a panel of islet cell autoantibodies.' In this high risk population, prior to or during the acute onset of clinical diabetes, substantial 13 cell mass may still be present such that if ongoing 13 cell-directed autoimmunity can be effectively and permanently inhibited, the remaining 13 cells may restore normoglycemia.^{2,3} Regulatory T cells (Tregs) play an important role in maintaining peripheral tolerance, and their deficiency has been associated with uncontrolled autoimmunity, including T1D.⁴ Therefore, immunotherapies directly or indirectly expanding Tregs have been viewed as a promising therapeutic approach.⁵⁻⁷ A recent phase 1 clinical trial demonstrated the feasibility of ex vivo expansion and the safety of adoptive transfer of polyclonal Tregs in T1D patients⁷, however efficacy of such adoptive immunotherapy with ex vivo expanded Tregs remains to be determined. On the other hand, antigen-specific Tregs are thought to be more potent than polyclonal Tregs in suppressing autoimmunity in T1D.^{8-1°} However, ex vivo expansion of antigen-specific Tregs for human therapies is highly labor-intensive and carries a significant regulatory and licensing burden, not to mention that the implicated 13 cell autoantigens are a moving target given the well-recognized epitope spreading in such individuals.” Consequently, immunotherapies aiming at in vivo expansion of endogenous Tregs are likely more feasible, and more likely to achieve the desired antigen-specific inhibition tailored to the specific set of autoantigens present in a given individual. The most promising antigen candidate for immunotherapies in T1D is insulin itself and its derivatives.¹² Ideally, a relevant insulin-derived autoantigen may be used to induce effective infectious tolerance' that spreads to other 13 cell autoantigens.

We and our colleagues have established an effective tolerogenic vaccine for controlling autoimmunity and alloimmunity.^{14,15} The tolerogenic vaccine is manufactured as antigen-coupled, ethylene carbodiimide (ECDI)-fixed splenocytes (Ag-SP), and is given via the intravenous (i.v.) route. Iv. injection of autoantigen-coupled Ag-SP has been shown to induce effective and long-lived antigen-specific tolerance in mouse models of autoimmune diabetes, 16 EAE17,18, allergic diseases¹⁹; and more recently by the Luo Lab in allogeneic and xenogeneic transplant models both in mice^{20,21} and in non-human primates (unpublished data). More importantly, a first-in-human clinical trial for multiple sclerosis based on this principle using myelin peptide-coupled autologous cells was recently published by our colleagues,²² establishing the clinical feasibility, safety and efficacy of this novel tolerance strategy. Interestingly, a prominent feature of Ag-SP-mediated tolerance is a robust in vivo expansion of endogenous Tregs,^{15,19,23} an observation that has recently been replicated in non-human primates.²⁴ Therefore, Ag-SP is a highly promising antigen-specific tolerance therapy for patients with T1D.

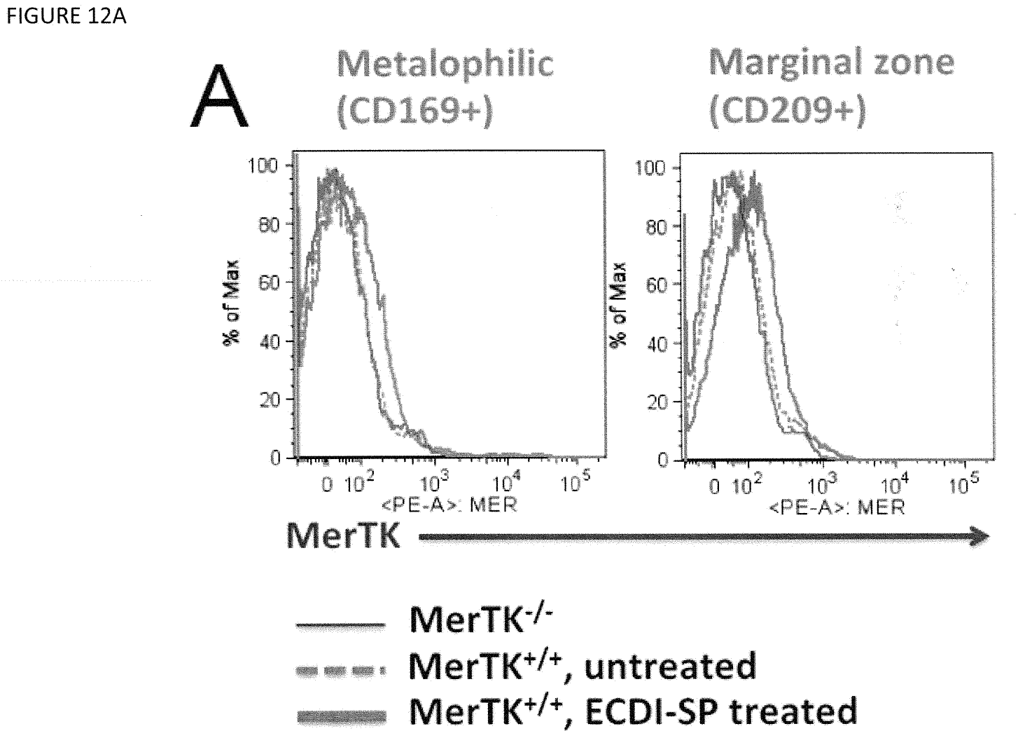

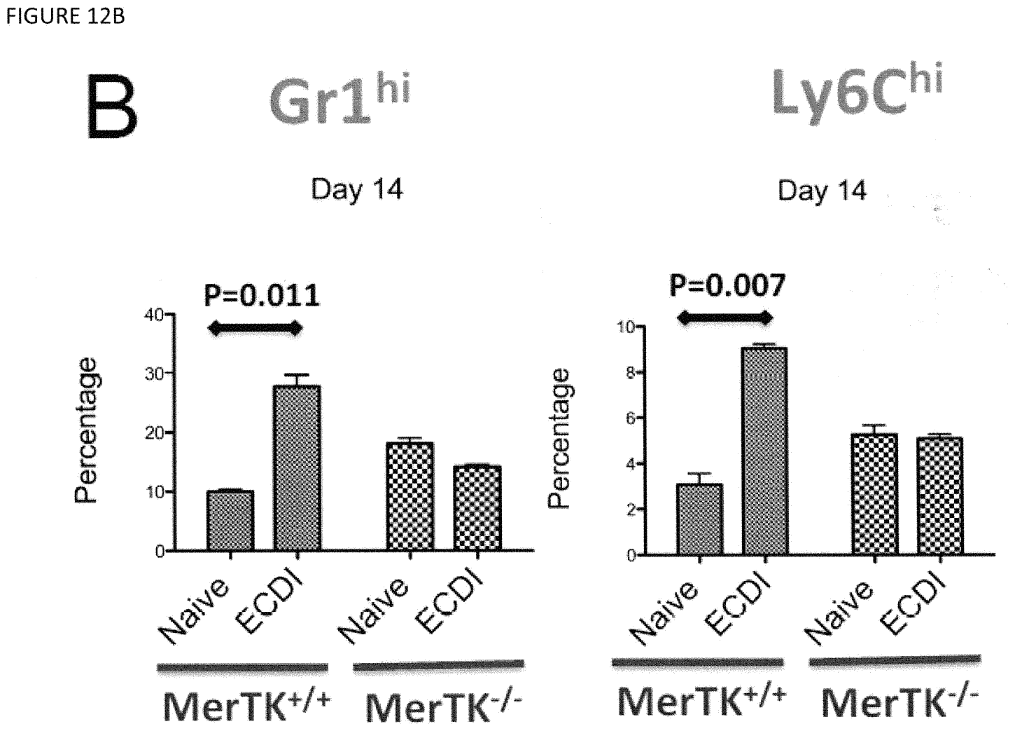

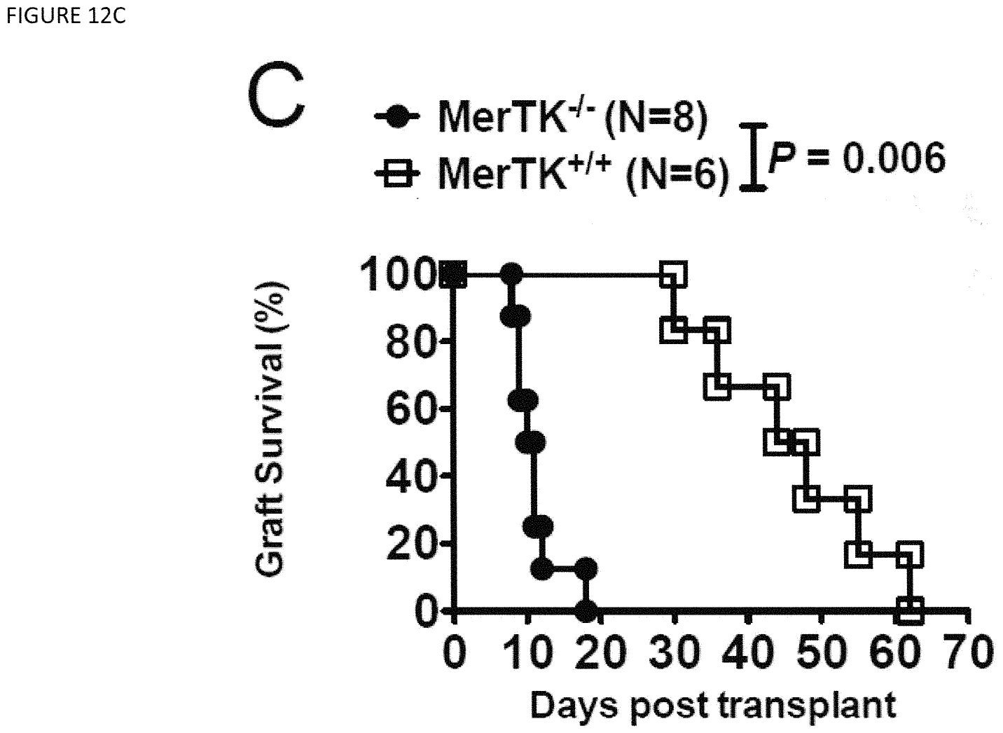

To circumvent the need for processing large numbers of patient cells formanufacturing Ag-SP, we have recently begun work using bioengineered nanoparticles (NPs) as carriers for the delivery of antigen cargos, and have published our early work demonstrating promising efficacy of such tolerogenic Ag-NP vaccines ²⁵⁻²⁷ However, in murine models of both food allergy and allogeneic islet transplant, we observe that such Ag-NP has a sub-optimal tolerance efficacy compared with Ag-SP. From these observations, we rationalize that there must exist additional tolerogenic signals provided by Ag-SP that are not present on Ag-NP. Through our preliminary studies, we identified two such missing tolerogenic signals from Ag-NP that can activate (1) the lectin CD209 and (2) the efferocytic receptor tyrosine kinase Mer (FIGS. 12A, B, and C) upon interacting with host phagocytes. Therefore, we hypothesize that conjugating the ligands for CD209 and Mer to NPs (e.g., via covalent attachment directly or indirectly via a linker) will significantly enhance the tolerogenicity of the Ag-NP vaccines.

Here, we propose to develop bioengineered NPs carrying CD209 and Mer dual signaling ligands, and test their ability to induce 13 cell-specific tolerance for T1D. Our compelling preliminary results, the comprehensive experimental plan, and the synergistic expertise of the research team present a unique opportunity for the design of a highly effective bioengineered Ag-NP vaccine for tolerance delivery in T1D.

Proposed Research

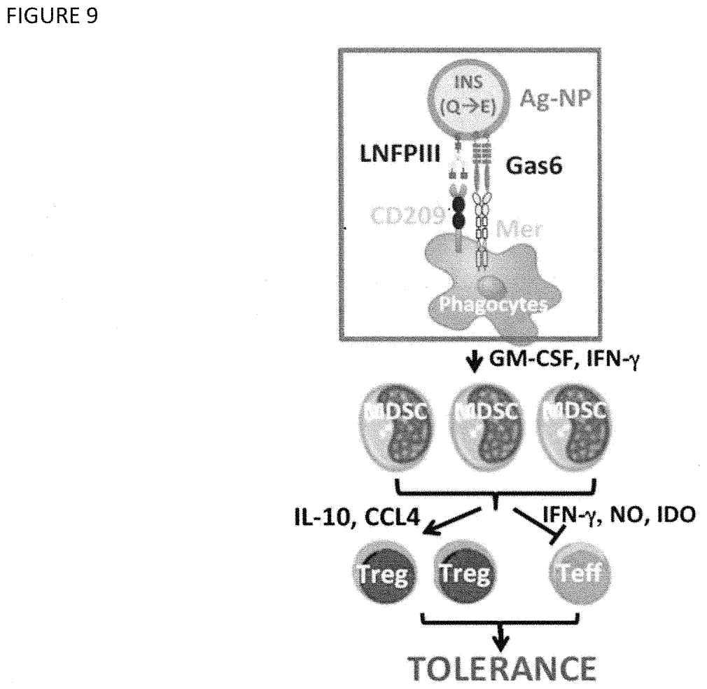

Central Hypothesis: As schematically shown in FIG. 9, we hypothesize that the efficacy of an Ag-NP delivery system for tolerance therapy in T1D can be significantly enhanced by: (1) simultaneous engineering targeting ligands (LNFPIII and GAS6) on NPs for CD209 and Mer dual signaling; and (2) delivery of the deamidated form of insulin (INS (Q-*E)) as the initial disease-relevant autoantigen for inducing infectious tolerance.

Specific Aims—Aim 1 To develop NPs comprising LNFPIII and GAS6 present on the surface of the NPs. Specifically, we will determine if conjugation of LNFPIII and GAS6 to NPs results in simultaneous targeting and signaling in appropriate murine phagocytes, leading to effective induction of tolerogenic features in these phagocytes. We hypothesize that LNFPIII-GAS6-NP effectively induces tolerogenic features in murine macrophages (MFs) via CD209 and Mer dual signaling. Aim 2 To test the tolerance efficacy of INS (Q 3E)-LNFPIII-GAS6-NP in the non-obese diabetic (NOD) mouse model. Specifically, we will determine in NOD mice, if delivery of LNFPIII-GAS6-NP coupled with deamidated mouse proinsulin results in robust tolerance to 13 cell-directed autoimmunity, and consequently prevents and/or reverses clinical diabetes in NOD mice. We hypothesize that INS(Q-*E)-LNFPIII-GAS6-NP effectively suppresses 13 cell-directed autoimmunity in NOD mice.

Rationale

Antigen-specific tolerance therapy has been the main focus of the Luo lab, particularly in the context of islet transplantation for T1D.¹⁴ Our primary approach has been to deliver (donor) antigens of interest by coupling them to the surface of splenocytes (Ag-SP) via amide bond formation in the presence of the carboxyl activating agent 1-ethyl-3-(3-dimethylaminoprophyl)carbodiimide (ECDI).^{2°} This approach was initially experimented by our colleagues for tolerance induction in animal models of autoimmunity.²⁸ Pioneering work in the Luo lab has further extended the robust efficacy of this tolerance approach to allogeneic and xenogeneic transplantation,^{21,23,29} while the Bryce lab has successfully demonstrated the efficacy of this approach in asthmatic and allergic disease.¹⁹ To circumvent the need for processing large numbers of patient cells for manufacturing Ag-SP, we have recently published our pioneering work using bioengineered NPs for tolerogenic antigen delivery, demonstrating promising efficacy of such tolerogenic Ag-NP vaccines.²⁵⁻²⁷ With a clear view of clinical translation, in this application we plan to focus our studies on developing a highly efficacious Ag-NP vaccine for tolerance delivery in T1D by: (1) binding CD209 and Mer dual signaling ligands to the surface of NPs; and (2) delivering deamidated proinsulin as the initial autoantigen for inducing infectious tolerance.

Rationale for LNFPIII and GAS6-mediated tolerance delivery: In murine models of both food allergy and allogeneic islet transplant, we observe that Ag-NP has a sub-optimal tolerance efficacy compared with Ag-SP. Through our preliminary studies, we identified two tolerogenic signaling receptors implicated in tolerance by Ag-SP that are not engaged by Ag-NP: (1) the efferocytic receptor tyrosine kinase Mer (FIGS. 12A, B, and C); and (2) the lectin CD209. Consequently, we hypothesize that binding the ligands for Mer and CD209 to the surface of NPs will significantly enhance the tolerogenicity of the Ag-NP vaccines. There are three members of the receptor tyrosine kinase (RTK) family that specialize in homeostatic clearance of apoptotic cells: TYR03, Axl and Mer, collectively called TAM RTKs, with the latter two being the principal TAM RTKs in the immune system.^{3°} Protein S and GAS6 (Growth Arrest-Specific are the two cognate ligands for TAM RTKs. TAM RTKs have two known functions: (1) to mediate “efferocytosis,” a process of homeostatic phagocytosis of apoptotic cells^{31,32}; and (2) to transmit regulatory signals that modulate innate immune responses. Deficiencies in TAM signaling are known to lead to profound autoimmunity.^{3°,33} Exogenous GAS6 can stimulate tyrosine autophosphorylation of both Mer and Axl, whereas Protein S is only capable of signaling through Mer.³⁵ In addition, GAS6 stimulates more efficient phagocytosis than Protein S,³⁵ particularly in setting of inflammation.³⁶ CD209 is a C-type lectin receptor present on the surface of MFs. Its signaling in MFs has been associated with IL-10-mediated suppressive functions of MF.³⁷ Lacto-N- fuconentaose III (LNFPIII) is a natural pentasaccharide containing the Lewis^{X} trisaccharide that binds and signals through CD209,³⁸ and has been shown to induce immunomodulatory effects,^{39,4°} prolong allograft survival⁴¹ and promote transplant tolerance.' In our preliminary studies (FIG. 12A), we observe that CD209 marks a prominent splenic MFs that upregulate the RTK Mer upon Ag-SP, but not Ag-NP, injection. These features make GAS6 and LNFPIII two attractive candidates for therapeutic bioengineering to Ag-NP for Mer and CD209 dual signaling, thus providing the missing signals that will enhance the tolerogenicity of Ag-NP.

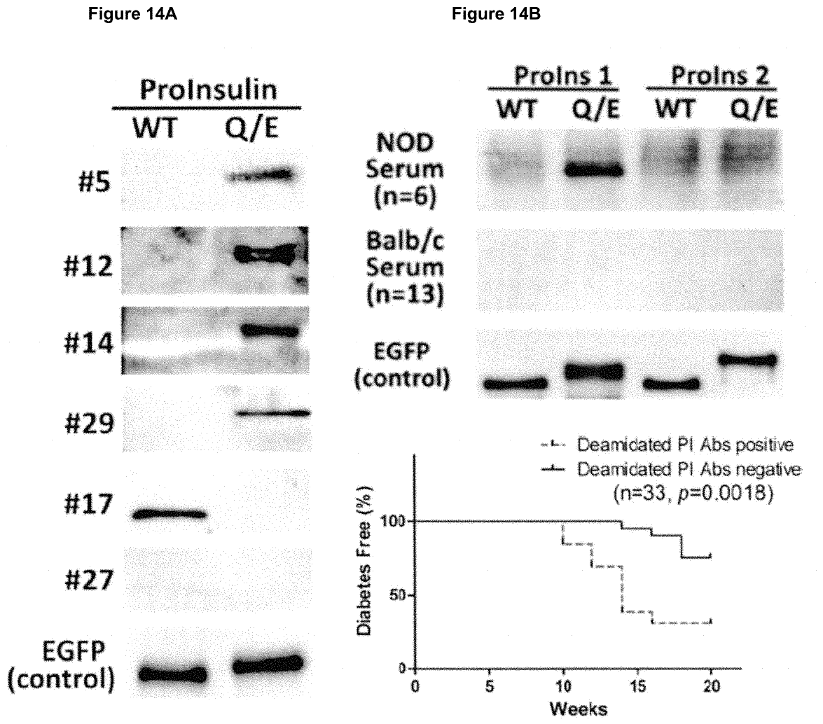

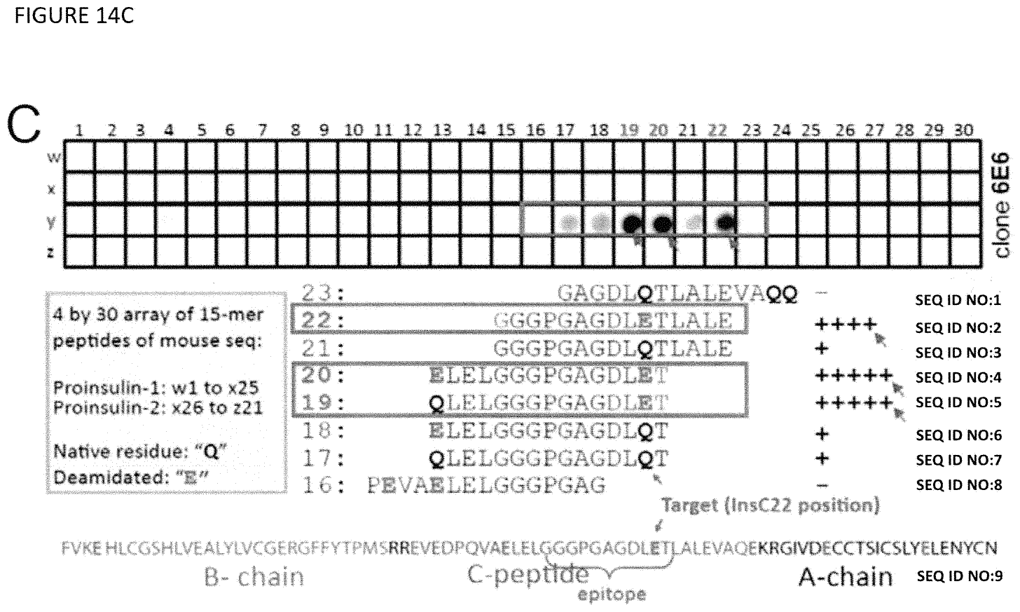

Rationale for deamidated insulin as the initial diabetes-relevant autoantigen to target: As islet 13 cells are highly susceptible to oxidative and ER stress under physiological conditions, proteins present in these cells have a high likelihood of undergoing various post-translational modifications (PTMs). Modified 13 cell proteins may generate neo-antigens that have not been self-tolerized through central and/or peripheral tolerance mechanisms, therefore are more likely to trigger immune responses and ensuing autoimmunity directed towards such neo-antigens. Three recent studies⁴²⁻⁴⁴ using cellular fractionation and mass spectrometry revealed that insulin is an abundant sources of polypeptide species generated by 13 cell secretory granules, consistent with existing literature demonstrating a prime role of insulin in mediating the autoimmunity against 13 cells.^{12,45} With a detailed examination of the Global Proteome Machine Database, we found that deamidation of glutamine (Q) is a frequent PTM on insulin. Deamidation of the side chain of Q can be catalyzed by the enzyme deamidase, or it can spontaneously occur when the protein is exposed to acidity. In vivo, 13 cells experiencing oxidative stress which causes vesicular acidification, conceivably providing an environment conducive for generating a large quantity of deamidated insulin protein/peptides. Strikingly, in our preliminary studies, we have detected stronger humoral (FIGS. 14A, B, and C) and cellular (data not shown) responses to deamidated (Q-*E) proinsulin than to native insulin, both in T1D patients and in NOD mice, confirming our hypothesis that such deamidated insulin is highly immunogenic. Importantly, such a response to deamidated proinsulin is significantly correlative to the incidence of diabetes development in NOD mice (FIG. 14B), a phenomenon currently also being evaluated in pediatric populations at risk for T1D. Given the more robust immune response towards deamidated proinsulin compared to native proinsulin, we hypothesize that tolerance will be more effective if deamidated proinsulin is used as a target antigen in our Ag-NP tolerance delivery approach. Intriguingly, cloning from hybridomas collected from NOD mice with positive humoral response to deamidated proinsulin and probing of peptide arrays allowed us to map the reactivity to a singular deamidated glutamine residue in the C-peptide (FIG. 14C). This highly immunogenic deamidated sequence of C-peptide will be our initial autoantigen candidate to target.

Preliminary Data

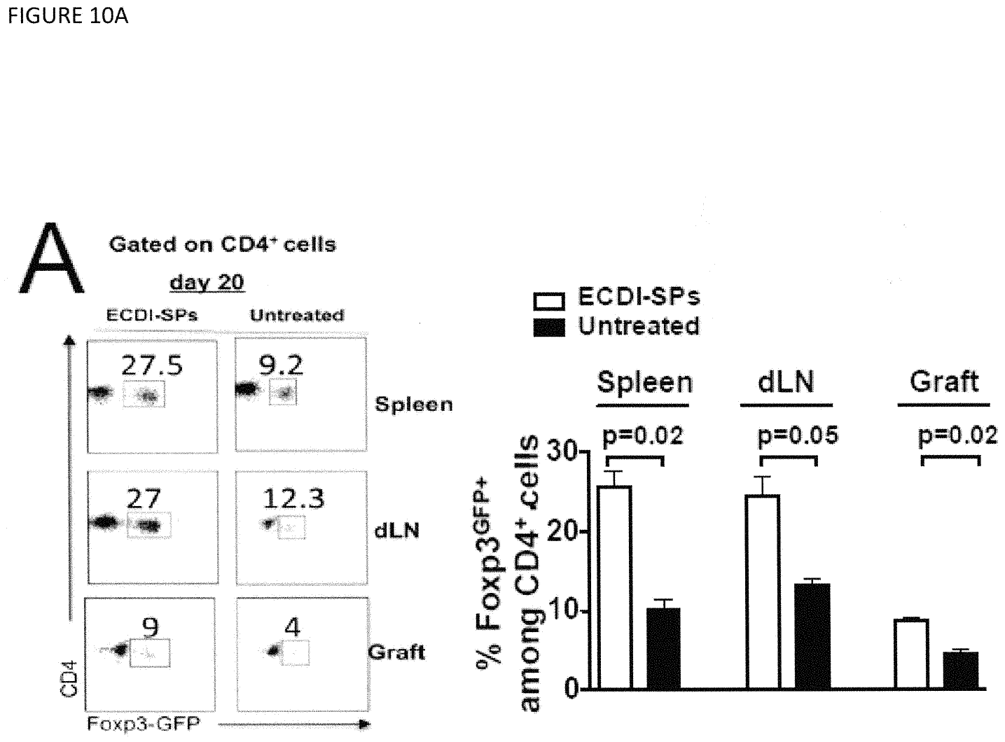

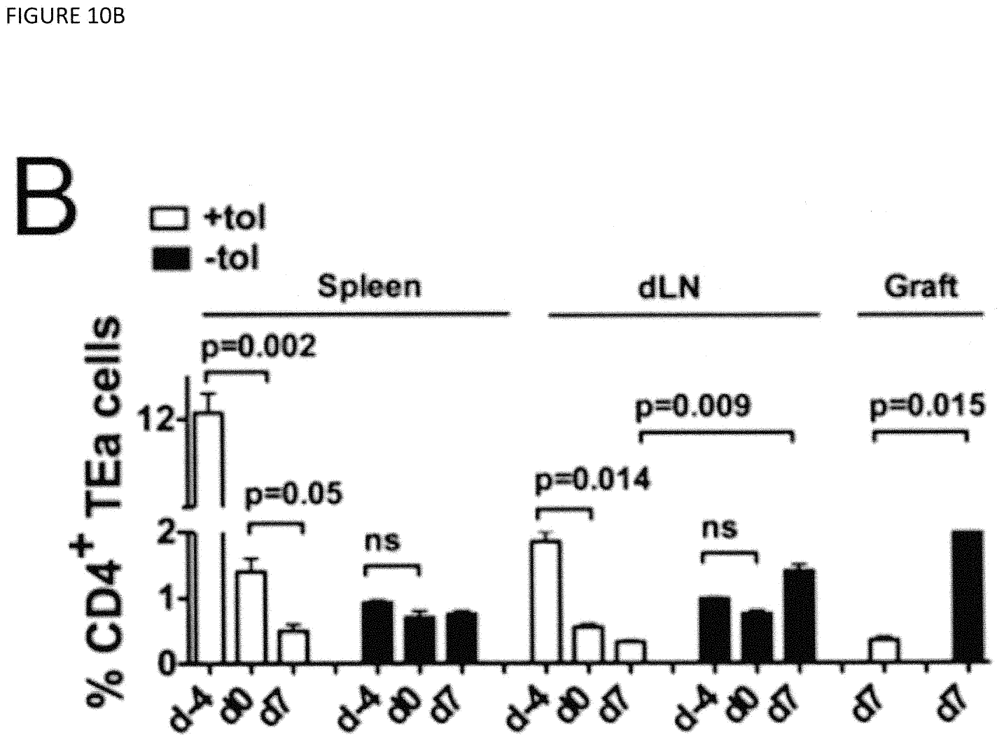

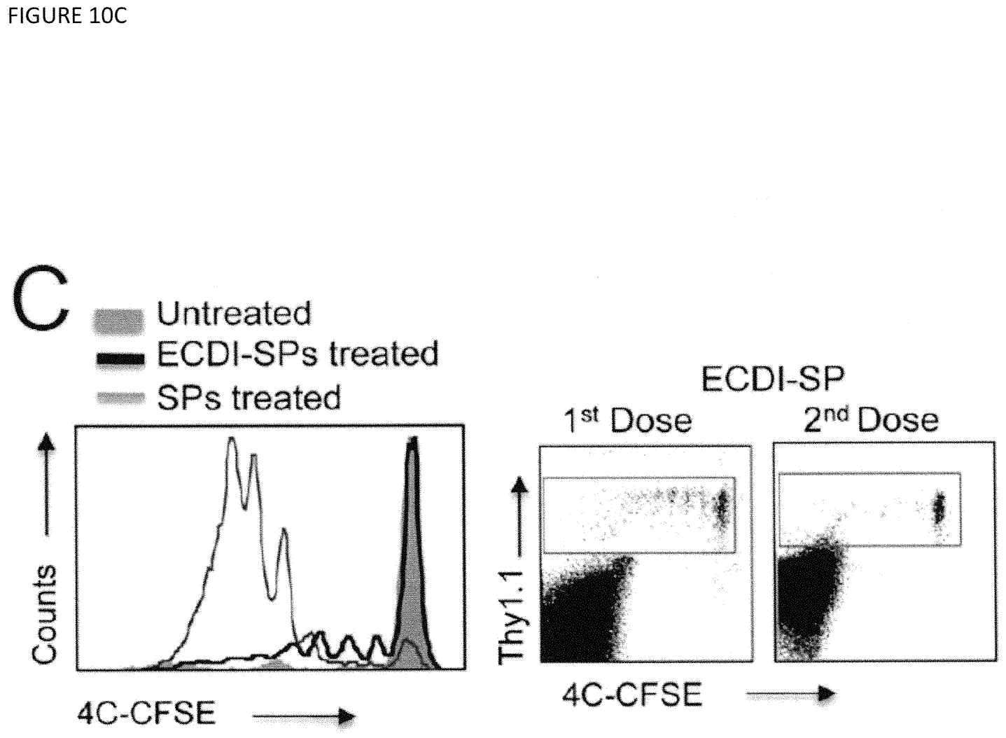

Antigen delivery via Ag-SP cells results in a significant expansion of Treg cells and tolerance of Teff cells via deletion and anergy. In a BALB/c 3 B6 allogeneic islet transplant model, injections of ECDI-fixed donor (BALB/c) splenocytes (Ag-SP) on day −7 and day +1 (with respect to BALB/c islet transplant on day 0) in B6 recipients result in indefinite islet allograft survival.²⁰ Ag-SP injections result a significant expansion of CD4⁺Foxp3⁺ Tregs in the spleen, draining lymph nodes (dLNs) and the transplanted allograft of the recipients (FIG. 10A). Accordingly, tolerance induced by Ag-SP is dependent on the expansion of Tregs by Ag-SP, as depletion of Tregs at the time of Ag-SP injections completely abrogated its tolerance efficacy.^{2°} Treg expansion by Ag-SP has recently been validated in a non-human primate islet allotransplantation model by our own work (unpublished data) and by published data of others.²⁴ Concomitant with Treg expansion, Teff cells are tolerized by two different mechanisms: (1) deletion of T cells with indirect specificity; and (2) anergy of T cells with direct specificity.²³ As shown in FIG. 10B, in the spleen and the dLNs, T cells with indirect donor specificity (interrogated by adoptive transfer of TEa TCR transgenic T cells⁴⁶) undergo a robust initial proliferation (day −4) followed by a rapid contraction and depletion (day 0, day 7), such that few such T cells infiltrate the islet allografts by day 7. In contrast, as shown in FIG. 10C, T cells with direct donor specificity (interrogated by adoptive transfer of 4C TCR transgenic T cells⁴⁷) undergo a significantly compromised proliferation to the first Ag-SP injection as compared to their proliferation to injection of untreated BALB/c SP. More importantly, the remaining 4C T cells no longer respond to donor stimulation as manifested in their lack of response to the second ECDI-SP injection (right dot plots), indicating that they are effectively anergized. Collectively, these data suggest that Ag-SP robustly expands Tregs while delete and/or anergize Teffs.

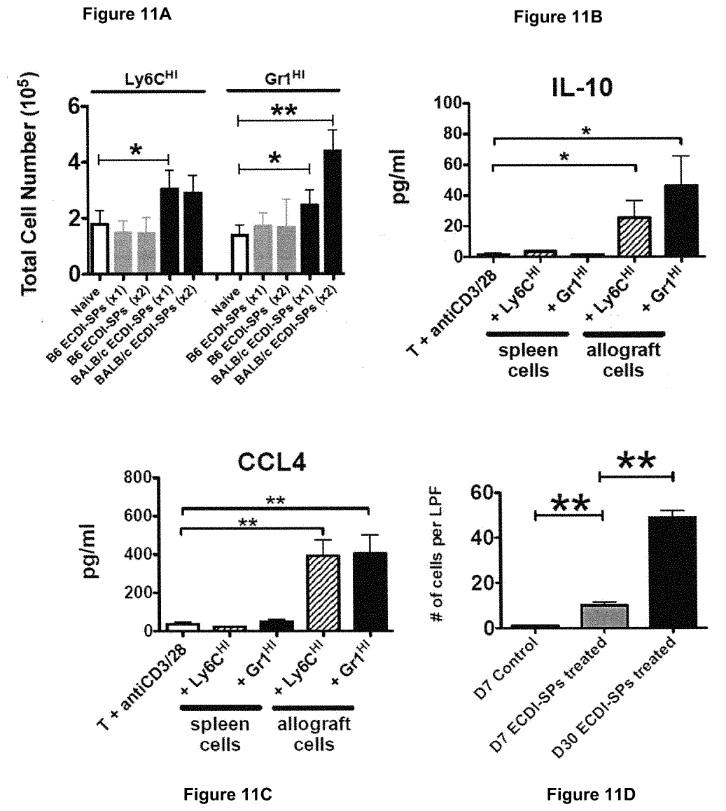

In Ag-SP tolerance, Treg induction and migration to site of inflammation is dependent on the expansion of myeloid derived suppressor cells (MDSCs). Ag-SP injections lead to significant expansions of two myeloid populations in the spleen (FIG. 11A) and the graft site (data not shown): the CD11b⁺Ly6C^{HI}Gr1^{1N4r} cells (referred to as Ly6C^{HI} cells) and the CD11b⁺Ly6C^{L°}Gr e^{ll} cells (referred to as Gri^{ll} cells). These two cell populations bear phenotypic resemblance to myeloid derived suppressor cells (MDSCs) and suppress T cell proliferation in vitro.^{29,48} Importantly, when co-cultured with T cells under anti-CD3/CD28 stimulation, allograft Ly6C^{HI} and Gr1^{m} cells are able to induce a significant production of IL-10 and CCL4, two soluble mediators implicated in the induction and homing of Treg cells (FIG. 11B). Supporting this possibility, allografts retrieved from donor Ag-SP-treated recipients show a progressive increase of Foxp3+ cells compared with those from control recipients (FIG. 11C). Consequently, depletion of Ly6C^{HI} and Gri^{m} MDSCs effectively abrogate tolerance induction by Ag-SP.^{29,48} Collectively, these data suggest that expansion of MDSCs is a critical step mediating Treg induction and migration induced by Ag-SP.

Expansion of Ly6C^{HI} and Gr1^{Ell} MDSCs by Ag-SP is dependent on the receptor tyrosine kinase Mer. When we tracked the injected Ag-SP in vivo, we found that they are retained in the splenic marginal zone and internalized by phagocytes in this region.²³ Because the receptor tyrosine kinase (RTK) family TAM (Tyro 3, Axl, Mer) has been implicated in homeostatic clearance of apoptotic cells, we first examined if they are implicated in tolerance induced by Ag-SP. As shown in FIG. 12A, Mer expression is induced by injections of Ag-SP, primarily on two splenic MF populations expressing cell surface lectins: the CD169⁺ transitional zone metallophilic MFs and the CD209⁺ marginal zone MFs. To determine if Mer induction on phagocyte populations plays a role in tolerance induced by Ag-SP, we took advantage of Mer mice. As shown in FIG. 12B, expansion of Ly6C^{HI} and Grlm MDSCs induced by Ag-SP is significantly blunted in Mer^{j-} mice.

Expansion of Ly6C^{HI} and Griₘ MDSCs by Ag-SP is dependent on the receptor tyrosine kinase Mer. When we tracked the injected Ag-SP in vivo, we found that they are retained in the splenic marginal zone and internalized by phagocytes in this region.²³ Because the receptor tyrosine kinase (RTK) family TAM (Tyro 3, Axl, Mer) has been implicated in homeostatic clearance of apoptotic cells, we first examined if they are implicated in tolerance induced by Ag-SP. As shown in FIG. 12A, Mer expression is induced by injections of Ag-SP, primarily on two splenic MF populations expressing cell surface lectins: the CD169⁺ transitional zone metallophilic MFs and the CD209⁺ marginal zone MFs. To determine if Mer induction on phagocyte populations plays a role in tolerance induced by Ag-SP, we took advantage of Mer^{-/-} mice. As shown in FIG. 12B, expansion of Ly6C^{HI} and Gri^{m} MDSCs induced by Ag-SP is significantly blunted in Mer^{-/-} mice induction of inhibitory monocytes and expansion of Treg cells 3a,3s,3˜,aI

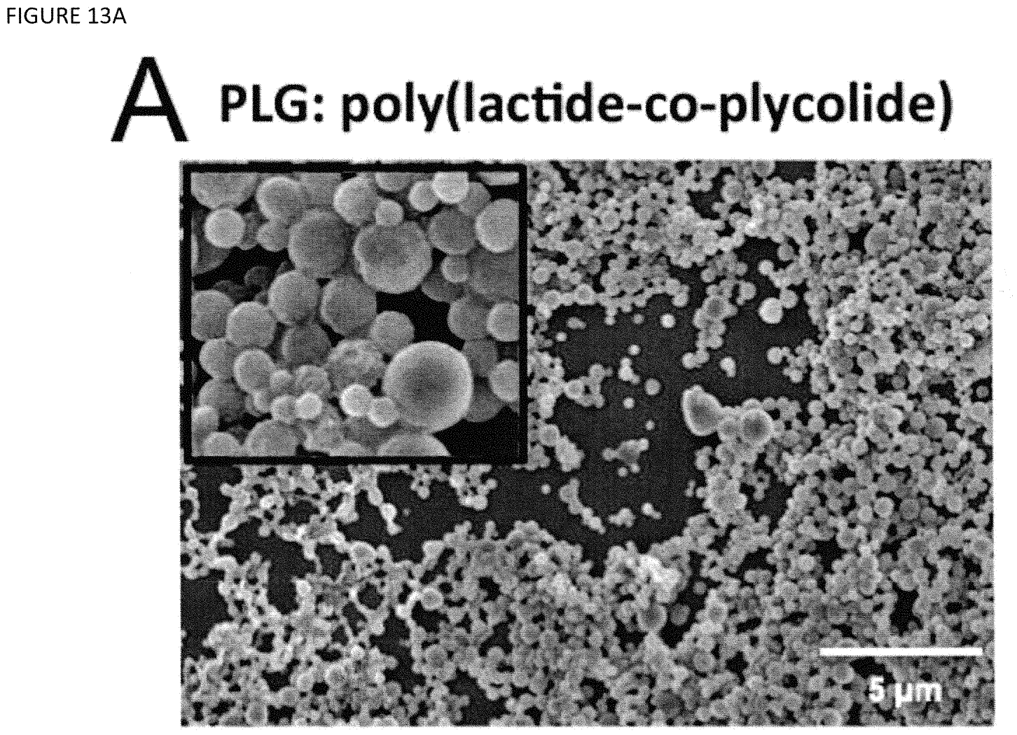

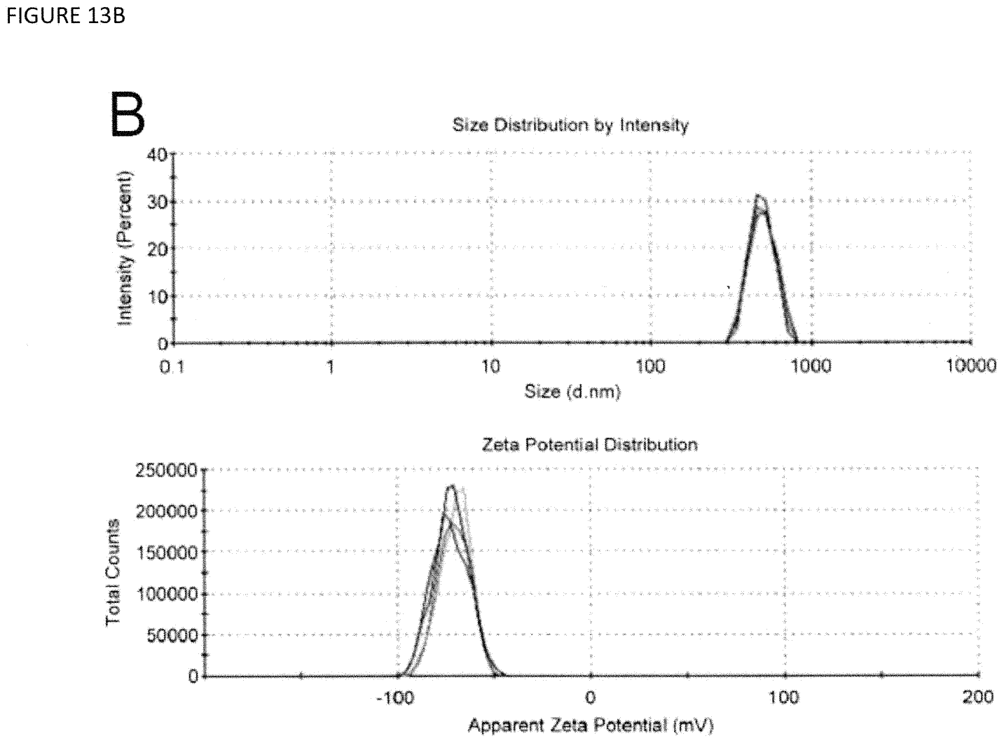

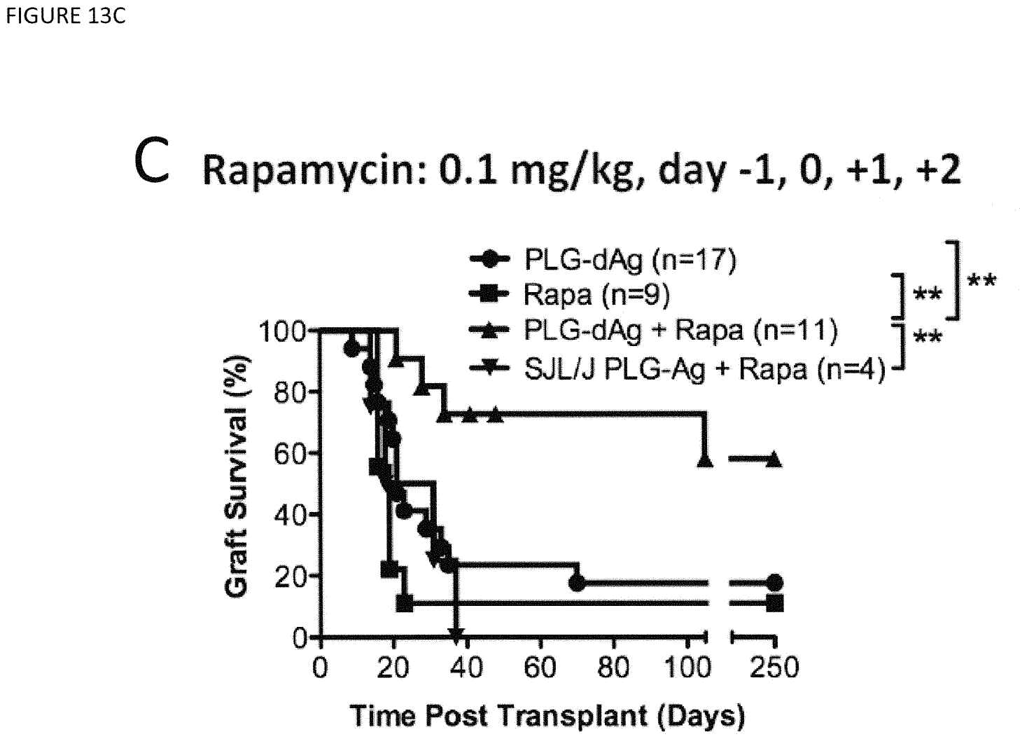

Nanoparticles (NPs) can be used for tolerogenic antigen delivery (Ag-NP). In an attempt to simplify and standardize antigen delivery, we and others have attempted to utilize PLG NPs as an antigen delivery vehicle.^{25-27,49} As shown in FIG. 13A, we manufactured PLG NPs with size and charge specifications, and coupled donor antigens (Ag) in the form of donor (BALB/c) splenocyte lysate using the same ECDI- coupling chemistry, and injected the Ag-NP to B6 recipients on day −7 and day +1, relative to BALB/c is let transplant on day 0. As shown in FIG. 13B, injections of Ag-NP alone (“PLG-dAg” group) resulted in only a marginal graft protection.Therefore, while the use of Ag-NP could greatly enhance the clinical feasibility of this tolerance approach, the tolerance efficacy via Ag-NP is less robust compared with that via Ag-SP.²⁵ We hypothesize that this is due to Ag-NP lacking critical cell surface carbohydrate and protein ligands, resulting in compromised tolerogenic signaling in the interacting phagocytes.

High-throughput screen demonstrates that carbohydrates can modulate the cytokine production repertoire of macrophages. In support of our overarching concept that ECDI-fixed NPs are lacking signals for induction of tolerogenic signals, we have examined the ability to modulate cytokine responses in a macrophage cell line (RAW264.7). This cell line has been used previously for predicting antigen presenting cell responses for tolerance. Using a 384-well high-throughput screen (HTS) based approach, whereby the cells are stimulated to express both IL-6 and IL-10 by LPS addition, ECDI-fixed SP led to significant increases in production of IL-10 (FIG. 1) and decreased IL-6 (not shown). In stark contrast, ECDI-treated NPs were ineffective and in fact reduced IL-10 production by RAW264.7 cells compared with the control (FIG. 1) Utilizing this HTS approach we examined an extensive range of potential signals that might be provided by cells but not NPs, and have focused on a panel of both natural and synthetic carbohydrate structures for their capacity to modulate the cytokine production repertoire of MFs. Based on a simultaneous enhancement of IL-10 and a suppression of IL-6 production, we successfully identified several such exciting carbohydrate candidates, including the Lewis^{X} antigen (FIG. 3A). Furthermore, in pilot experiments, the attachment of fucose was sufficient to enhance NP uptake by the cells and promote an IL-10-skewed response (FIG. 5).

Proinsulin Q3E deamination elicits robust immune response in both humans and mice. We first synthesized recombinant human proinsulin or mouse proinsulin 1 and proinsulin 2 proteins with all of their glutamine (Q) residues mutated to glutamate (E) residues, and used these Q-*E proinsulin proteins to probe sera of a cohort of 30 adult patients with known T1D (FIG. 14A) and 33 young NOD female mice starting at 3 weeks of age (FIG. 14B). As shown in FIG. 14A, four out of the 30 adult T1D patients had an antibody response to the deamidated proinsulin but not to the native (WT) proinsulin. Similarly, as shown in FIG. 14B top panel, individual NOD mice developed an antibody response to the deamidated proinsulin but not to the WT proinsulin (shown is an example of antibody response to deamidated murine proinsulin 1, but response to deamidated murine proinsulin 2 or both was also observed). Importantly, in NOD mice, the humoral response to deamidated proinsulin is highly correlative to the incidence of diabetes development (FIG. 14B bottom panel). This predictive correlation is currently also being evaluated in pediatric populations at risk for T1D. Intriguingly, cloning from hybridomas collected from NOD mice with positive humoral response to deamidated proinsulin and probing of peptide arrays as exemplified in FIG. 14C allowed us to map the humoral reactivity to a singular deamidated glutamine residue within the C-peptide: spot Y19, 20, 22, correlating to the sequence GGGPGAGDLET (SEQ ID NO:4).

Research Design and Methods

Aim 1 To develop LNFPIII and GAS6 decorated NPs

Aim 1A. Design and manufacturing of LNFPIII-GAS6-NP. We will first fabricate poly(lactide-co-glycolide (1:1)) (PLG) nanoparticles, approximately 500 nm in diameter, using the single emulsion technique as previously described by Bryant et al.²⁵ The surface of the nanoparticles will be partially hydrolyzed with 0.05 or 0.1 M NaOH to increase the density of carboxyl groups available for functionalizing the surface of the particles and coupling the antigens. The modification will be monitored by measuring the NP zeta potential as well as quantifying the carboxyl content using toluidine blue.^{5°} The carboxyl groups on the NPs will be activated using carbodiimide chemistry (ECDI) and reacted with (N-maleimidopropionic acid hydrazide (BMPH) in order to provide maleimide groups on the surface of the NPs, which are reactive toward thiol groups utilized in “click” chemistry.' The ligands LNFPIII and GAS6 will be derivatized with cysteine to provide the thiol group that will allow their covalent linkage to the maleimide-functionalized NPs. LNFPIII-Cys will be synthesized via reductive amination between LNFPIII and Cys.⁵² The GAS6 with a terminal Cys will be synthesized via recombinant DNA technology using a His6 tag in HEK 293T cells and isolated via affinity chromatography with Ni-NTA beads followed by purification on a HiTrap Q FF ion exchange column (GE Healthcare) as previously described.

Both LNFPIII-Cys and GAS6-Cys will be attached to the PLG-NPs via click chemistry.⁵¹ If the expected results are not obtained using click chemistry as determined by the RAW264.7 MF assay (described in detail in Aim 1B below), alternatively the PLG NPs will be functionalized with streptavidin via carbodiimide chemistry. The streptavidin-PLG NPs will be subsequently reacted with biotinylated LNFPIII and GAS6. Coupling efficiencies of the ligands will be determined by quantifying protein and carbohydrate in the supernatants before and after the coupling reaction. Furthermore, the protein and carbohydrate on the surface of the NPs will be detected via labeled antibodies that are specific for GAS6 and LNFPIII.

If activation using the PLG platform is suboptimal as determined by the RAW264.7 MF assay (described in detail in Aim 1B below), we will investigate the use of poly(polyethylene glycol citrate-co-N-isopropylacrylamide) (PPCN) as a delivery platform. PPCN is a thermoresponsive biodegradable macromolecule developed that has been shown to be biocompatible and capable of slowly delivering proteins.⁵³ This macromolecule has a high density of carboxyl groups that can be functionalized and can easily form NPs of approximately 200-300 nm in diameter under very mild conditions. The ligands can be conjugated to PPCN using the same click chemistry described above for PLG NPs. A potential advantage of using PPCN is the display of a significantly higher density of ligands on the surface of NPs due to direct conjugation of the macromolecule to the ligands and the formation of the NPs via self-assembly of the ligand-functionalized PPCN.

Aim 1B. Screening of LNFPIII-GAS6-NP by cytokine modulation in RAW264.7 MFs. The LNFPIII-GAS6-NP developed as in Aim lA will be screened using a co-culturing system with RAW264.7 cell line macrophages as shown in FIG. 2. We anticipate that LNFPIII-GAS6-NP with variable parameters (conjugating methods (click chemistry vs. biotin-streptavidin), polymer materials (PLG vs. PPCN)) will be sequentially manufactured and therefore will be tested on a rolling basis. Each species of LNFPIII-GAS6-NP will be co-cultured with RAW264.7 MFs in the presence of LPS stimulation (MFs +LNFPIII-GAS6-NP+LPS) for 72 hours. Resulting supernatants will be measured for IL-10 and IL-6 by ELISA. Control co-cultures will include: (1) MFs alone; (2) MFs+LPS; (3) MFs +unmodified NP; (4) MFs+unmodified NP+LPS; and (5) MFs+LNFPIII-GAS6-NP. The IL-10/IL-6 ratio of control condition #2 will be considered as the baseline. An IL-10/1L-6 ratio above the baseline will be considered screened “positive”; whereas a ratio below the baseline will be considered screened “negative.” To collaterally support results obtained from RAW264.7 MF cell line, screened “positive” LNFPIII-GAS6-NP species will also be confirmed using primary murine bone marrow derived MFs in a similar co-culturing system.

Aim 1C. Antigen loading to screen “positive” LNFPIII-GAS6-NP. We will load three possible 13 cell antigens for experiments proposed in Aim 2deamidated proinsulin peptide “GGGPGAGDLETLALE (SEQ ID NO:2)” (FIG. 14C), deamidated whole proinsulin, or whole MING ((3 cell line) cell lysate. We will test two methods for antigen loading to the screen “positive” LNFPIII-GAS6-NP species. The first method will be coupling peptide/protein antigens to the surface of the nanoparticles using the ECDI chemistry as we previously described.²⁵ The amount of peptides/proteins coupled to the particles will be determined by quantifying the antigens in the supernatants before and after the coupling reaction. If either the coupling efficiency or their interaction with RAW264.7 MFs is suboptimal, we will also test if encapsulation of antigens within the particles will be more efficient. PLG particles formed with encapsulated peptides have been effective in models of autoimmune encephalitis.” The antigens will be encapsulated into PLG or PPCN particles via a double emulsion process that aims to create particles with similar diameter and charge as the single emulsion process (500 nm, C potential=−60 mV). Polymer compositions and average molecular weights (as characterized by inherent viscosity) for encapsulation will be experimented, as these properties will influence the stability of the NPs, therefore influence both cellular internalization and release of the encapsulated antigens.⁵⁵ The distribution of particle sizes and the zeta potential will be measured with a zetasizer. The amount of peptides/proteins encapsulated within the particles will be quantified by dissolving the antigen-loaded NPs in DMSO for subsequent analysis with a CBQCA assay.⁵⁶

[Expected outcome, potential pitfalls and alternative approaches. We anticipate that conjugating LNFPIII and GAS6 to NPs will significantly enhance their tolerogenic interaction with MFs and lead to a favorable IL-10/IL-6 production ratio. We anticipate that with varying conjugating methods (click chemistry vs. biotin-streptavidin), polymer materials (PLG vs. PPCN), methods for antigen loading (crosslinking vs. encapsulation), choice of 13 cell antigens (proinsulin peptide vs. whole protein vs. whole (3 cell lysate), we will generate a library of Ag-LNFPIII-GAS6-NP species with a spectrum of MF IL-10/IL6 production ratio. The top performers will be selected for experiments proposed in Aim 2 If suboptimal IL-10/IL-6 production ratio is observed across the board with all variations, one additional consideration is to enhance GAS6 signaling by linking phosphatidylserine (PS) to the GLA domain of GAS6.³² This can be accomplished by incorporation of PS onto PLG or PPCN particles by an emulsion process with the addition of PS at a weight ratio of 1:10 (PS:polymer).⁵⁷ PS has a carboxylic acid head group and alkyl tails, therefore possessing functional groups for both incorporation onto polymer particles and for antigen loading via ECDI coupling or encapsulation.

Aim 2 To test the tolerance efficacy of 1NS(Q3E)-LNFPIII-GAS6-NP in the NOD mouse model

Aim 2A: Prevention and treatment of diabetes in NOD by tolerogenic INS(Q-*E)-LNFPIII-GAS6-NP vaccines. Mouse Models. We will use two NOD models. In the first model (the “prevention” model), we will treat two age cohorts: 5-week old and 9-week old_female NOD mice. In both age groups, the inflammatory responses in the pancreas have already begun as demonstrated by the presence of pro-inflammatory immune cell infiltration, but the blood glucose levels are still within normal range. Thus they are pre-diabetic. We will apply the best identified formula of INS(Q-*E)-LNFPIII-GAS6-NP treatment (by Aim 1) to determine if we can prevent these pre-diabetic NOD mice from developing diabetes.The mice will be monitored following the INS(Q-*E)-LNFPIII-GAS6-NP treatment for blood glucose levels until 30 weeks of age. In the second model (the “treatment” model), we will use acute diabetic (12-30-week old) NOD mice that have just become hyperglycemia identified by twice a week screening starting at age 12 weeks. We will apply the INS(Q-*E)-LNFPIII-GAS6-NP treatment within 3-5 days of acute onset of hyperglycemia. At this stage, significant 13 cell mass still remains in these NOD mice such that effective control of autoimmunity with immunotherapy can lead to recovery of function of the remaining 13 cells and consequently reversal of diabetes.⁵⁸ We will apply the INS(Q-*E)-LNFPIII-GAS6-NP treatment to the acutely diabetic NOD mice and determine if we can reverse the diabetes in these mice. The mice will be monitored for blood glucose levels for a total 60 days following the INS(Q-*E)-LNFPIII-GAS6-NP treatment to determine diabetes reversal.

1NS(Q3E)-LNFPIII-GAS6-NP treatment. NP species with a robust IL-10/1L-6 production ratio upon co-culturing with RAW264.7 MFs will be manufactured in therapeutic quantities for loading the targeted antigen for in vivo treatment of NOD mice. Initially, we will test the 15-aa proinsulin peptide “GGGPGAGDLETLALE” (SEQ ID NO:2) containing the critical site of deamidation identified as in FIG. 14C as our targeted antigen. The 15-aa INS(Q-*E) peptide will be either attached to the surface of the LNFPIII-GAS6-NP (via ECDI-mediated crosslinking⁵⁴) or encapsulated within the LNFPIII-GAS6-NP. The choice between crosslinking and encapsulation will be determined based on antigen loading efficiency as determined in Aim 1C. 3 mg of INS(Q-*E)-LNFPIII-GAS6-NP will be injected i.v. to female NOD mice of the three age groups (5-week, 9-week or acute diabetic). Control mice will be age-matched female NOD mice injected with LNFPIII-GAS6-NP loaded with the native proinsulin peptide (“GGGPGAGDLQTLALE” (SEQ ID NO:3)), unloaded LNFPIII-GAS6-NP, or no NPs. These control groups will allow us to determine if: (1) naked LNFPIII-GAS6-NP will have any disease modifying effect themselves as has been described in CNS infection and cardiac ischemia models²⁷; and (2) targeting deamidated proinsulin peptide is more effective than targeting the native proinsulin peptide. If the experimental groups (treated with INS(Q-*E)-LNFPIII-GAS6-NP) demonstrate a superior diabetes control, we will also test if multiple injections of the effective INS(Q-*E)-LNFPIII-GAS6-NP vaccine every 4 weeks will have additional benefit for sustained disease control. In order to complete our proposed experiments within the timeline of this grant (2 years), we anticipate that we will need to test on a rolling basis multiple promising NP species as they are developed and validated to fulfill the IL-10/1L-6 readouts as defined above.

Additional autoantigens to be tested as tolerogenic LNFPIII-GAS6-NP vaccines for T1D. In addition to the “GGGPGAGDLETLALE” (SEQ ID NO:2) proinsulin peptide, it is possible that a pool of multiple autoantigens will need to be included achieve effective tolerance,^{22,59} particularly during later stages of the disease when auto-antigenicity may have spread to other epitopes. Therefore, if INS(Q-*E)-LNFPIII-GAS6-NP exhibit disease “breakthrough,” especially in older mice, we will deliver additional possible autoantigens using the same LNFPIII-GAS6-NP vehicle. The additional possible autoantigens to be tested via tolerogenic LNFPIII-GAS6-NP delivery are: (a) Deamidated whole insulin: Because whole insulin in its native form has been shown to have demonstrated efficacy in tolerance therapies in NOD mice^{16,59} and our preliminary result (FIG. 14B) demonstrated a heightened immune response to deamidated whole proinsulin, we will also test deamidated whole insulin (recombinant mouse proinsulin 1 and proinsulin 2 proteins with all of their glutamine (Q) residues mutated to glutamate (E) residues) as the autoantigens delivered by LNFPIII-GAS6-NP to determine if such deamidated proinsulin with a broadened scope of epitopes exhibit better tolerance efficacy compared to that of the “GGGPGAGDLETLALE” (SEQ ID NO:2) alone. (b) Whole 13 cell lysate: We will prepare whole 13 cell lysate from the insulinoma cell line MING derived from transgenic mice expressing the large T-antigen of SV40 in their 13 cells.^{6°} Whole 13 cell lysate will be either attached to the surface via ECDI crosslinking (as we have previously done with donor cells lysate for transplant antigens²⁵) or encapsulated within the LNFPIII-GAS6-NP. The choice between crosslinking and encapsulation will be similarly determined based on antigen loading efficiency as described in Aim 1C. 13 cell lysate-LNFPIII-GAS6-NP will be injected to prediabetic and acute diabetic female NOD mice, and mice will be monitored for diabetes prevention and diabetes reversal respectively.

Experimental readouts. For the diabetes prevention group (pre-diabetic NOD mice), blood glucose levels will be checked twice a week following the INS(Q-*E)-LNFPIII-GAS6-NP treatment until the mice reach 30 weeks of age. The percentage of mice developing diabetes will be compared with that of control groups. For the diabetes treatment group (diabetic NOD mice), blood glucose levels will be check twice a week following the INS(Q-*E)-LNFPIII-GAS6-NP treatment for a total of 60 days. The percentage of mice restoring normoglycemia will be compared with that of control groups. At the termination of the experiment, NOD mice will be sacrificed for examination of the pancreas of islet size, number and architecture, and infiltration of inflammatory cells.

Aim 2B: Determine the mechanisms of protection by tolerogenic 1NS(Q3E)-LNFPIII-GAS6-NP vaccines. Expansion of MDSCs. We will examine the effect of INS(Q-*E)-LNFPIII-GAS6-NP vaccines on in vivo expansion of MDSCs and Tregs, and inhibition of Teffs. Treated and control NOD mice will be examined for expansion of CD11b⁺Ly6C^{HI}Gr1^{INT} (LyC^{ill}) cells and CD11b⁺Ly6C¹⁻ⁿGr1^{ill} (Gr1)cells in the spleen and the pancreas. Ly6C^{HI} or Gri^{m} cells isolated from the spleen and the pancreas of treated and control NOD mice will be co-cultured with naive NOD T cells stimulated by anti-CD3/CD28 for 72 hours. Suppression of T cell proliferation will be determined by CFSE dilution. Production of IL-10 and CCL4 in culture supernatant will be measured by ELISA as shown in FIG. 11B, and expansion of Tregs will be determined by enumerating Foxp3⁺ cells following co-culturing with Ly6C^{III} or Gr1^{m}.

Expansion of autoantigen-specific CD4⁺Foxp3⁺Tregs. Treated and control NOD mice will be examined for the induction or the expansion of antigen-specific CD4⁺Foxp3⁺ Tregs with specificities towards the modified proinsulin peptide “GGGPGAGDLETLALE” (SEQ ID NO:2): (a) the pancreatic DLN and the spleen will be examined (by FACS) for total number of CD4⁺Foxp3⁺ Tregs at serial time points following INS(Q-*E)-LNFPIII-GAS6-NP treatment; (b) purified total CD4⁺ T cells (Tregs and non-Tregs) from the pancreatic DLN or the spleen will be stimulated with the “GGGPGAGDLETLALE” (SEQ ID NO:2) peptide, or an irrelevant OVA peptide, or anti-CD3 antibody (pan-TCR stimulation). Post-stimulation, CD4⁺Foxp3⁺ Tregs will be enumerated to determine if expansion of Tregs has occurred in an antigen-specific manner. (c) enriched CD4⁺CD25 T cells (non-Tregs) from the pancreatic DLN or the spleen will be stimulated with the same “GGGPGAGDLETLALE” (SEQ ID NO:2) peptide, or an irrelevant OVA peptide, or anti-CD3 antibody (pan-TCR stimulation). Post-stimulation, CD4⁺Foxp3⁺ T cells will be enumerated to determine if induction of Tregs has occurred in an antigen-specific manner.

] Inhibition of autoantigen-specific effector T cells (Teff). Treated and control NOD mice will be examined for autoantigen-specific Teff cell function as follows: (a) the pancreatic DLN and the spleen will be examined and enumerated (by FACS) for CD4 or CD8, IFN-γ, or IL-17 producing cells at serial time points following INS(Q-*E)-LNFPIII-GAS6-NP treatment; (b) enriched total CD4⁺ T cells (Tregs and non-Tregs) from the pancreatic DLN or the spleen will be stimulated with “GGGPGAGDLETLALE” (SEQ ID NO:2) peptide, or an irrelevant OVA peptide, or anti-CD3 antibody (pan-TCR stimulation). Post-stimulation, T cell proliferation will be determined by CFSE dilution, and T cell-derived proinflammatory cytokines including IFN-y, IL-17, and IL-4 will be determined by ELISA assay of the culture supernatant; (c) purified CD4⁺CD25 T cells (non-Tregs) from the pancreatic DLN or the spleen will be stimulated with the “GGGPGAGDLETLALE” (SEQ ID NO:2) peptide, or an irrelevant OVA peptide, or anti-CD3 antibody (pan-TCR stimulation). Post-stimulation, T cell proliferation and T cell-derived cytokines in the absence of Tregs will be measured to determine if proliferation and/or inflammatory cytokine production is increased back to the level of T cells from untreated mice.

Expected outcome, potential pitfalls and alternative approaches. We anticipate that diabetes will be prevented in pre-diabetic NOD mice and reversed in acute diabetic NOD mice treated with the INS(Q-*E)-LNFPIII-GAS6-NP vaccine. Furthermore, we anticipate that deamidated proinsulin or proinsulin peptide will be more effective at inducing tolerance than their unmodified counterpart. Finally, at late stages of diabetes, tolerance using a broader antigen pool such as whole 13 cell lysate may be more effective than single protein/peptide vaccine alone. Protected NOD mice will exhibit preserved islet architecture and diminished insulitis. We also anticipate that a higher number of Tregs demonstrating autoantigen specificity will be observed in NOD mice treated with the tolerogenic INS(Q-*E)-LNFPIII-GAS6-NP vaccine. Conversely, autoantigen-stimulated, but not nonspecific anti-CD3 stimulated, effector T cell proliferation and proinflammatory cytokine production will be inhibited in treated mice, and this inhibition is Treg-dependent. We predict that the tolerogenic INS(Q-*E)-LNFPIII-GAS6-NP vaccine reprograms the immune system by dually inducing autoantigen-specific Tregs and inhibiting autoantigen-specific Teffs. It is expected that findings and knowledge acquired from aforementioned experimental studies would provide mechanistic and practical foundations for translating our approach to clinical settings for patients with T1D. If INS(Q-*E)-LNFPIII-GAS6-NP vaccines demonstrate promising efficacy in controlling autoimmunity during pre-diabetic and acute diabetic stages, future studies beyond the two year proposed funding period will be designed to further examine: (1) late diabetic stages by using the NOD syngeneic islet transplant model as we previously published⁵⁸; (2) the induction of infectious tolerance by examining tolerance of T cells with other antigen specificities (such as NOD 8.3⁶¹ (specific to IGRP) or NOD BDC2.5⁴⁵ (specific to ChgA) T cells) by the insulin-specific 1NS(Q3E)-LNFPIII-GAS6-NP vaccine. If INS(Q-*E)-LNFPIII-GAS6-NP vaccines demonstrate only partial efficacy in NOD mice, we will consider combinatorial therapies, such as additional low dose IL-2 or rapamycin, that might further tip the balance of the Treg/Teff towards regulation.

Advantages over alternative approaches that would address our goals. Current antigen-specific immunotherapies for T1D comprise largely of antigens only, therefore have limited potency. Our approach of delivering 13 cell neo-autoantigens via LNFPIII-GAS6-NP will provide targeted tolerogenic signals to host phagocytes, expand endogenous suppressor cell populations such as MDSCs and antigen-specific Tregs, and ultimately enhance tolerance efficacy and yet preserve the simplicity of the manufacturing of the Ag-NP vaccine. In addition, it offers a platform technology that has the potential for a wide applicability to other autoimmune and allergic conditions.

Should our JDRF grant proposal be funded, it is extremely likely that the proposed research will lead to the establishment of an industry collaboration with a focus on the development and licensing of a T1D therapeutic product. Within the first year of the proposed funding period, we will likely have obtained sufficient preliminary data on the manufacturing and therapeutic efficacy of the INS(Q-*E)-LNFPIII-GAS6-NP tolerogenic vaccine to engage an industry partner.

### REFERENCES

1. Brorsson C A, Onengut S, Chen W M, et al. Novel Association Between Immune-Mediated Susceptibility Loci and Persistent Autoantibody Positivity in Type 1 Diabetes. Diabetes 2015; 64:3017-27.

2. Voltarelli J C, Couri C E, Stracieri A B, et al. Autologous nonmyeloablative hematopoietic stem cell transplantation in newly diagnosed type 1 diabetes mellitus. JAMA: the journal of the American Medical Association 2007; 297:1568-76.

3. Moole H, Moole V, Mamidipalli A, et al. Spontaneous complete remission of type 1 diabetes mellitus in an adult-review and case report. J Community Hosp Intern Med Perspect 2015; 5:28709.

4. Sakaguchi S. Naturally arising Foxp3-expressing CD25+CD4+ regulatory T cells in immunological tolerance to self and non-self. Nature immunology 2005; 6:345-52.

5. Tan T, Xiang Y, Chang C, Thou Z. Alteration of regulatory T cells in type 1 diabetes mellitus: a comprehensive review. Clin Rev Allergy Immunol 2014; 47:23443.

6. Pham M N, von Herrath M G, Vela J L. Antigen-Specific Regulatory T-Cells and Low Dose of IL-2 in Treatment of Type 1 Diabetes. Frontiers in immunology 2015; 6:651.

7. Bluestone J A, Buckner J H, Fitch M, et al. Type 1 diabetes immunotherapy using polyclonal regulatory T cells. Science translational medicine 2015; 7:315ra189.

8. Tang Q, Henriksen K J, Bi M, et al. In vitro-expanded antigen-specific regulatory T cells suppress autoimmune diabetes. The Journal of experimental medicine 2004; 199:1455-65.

9. Tarbell K V, Yamazaki S, Olson K, Toy P, Steinman RM. CD25+CD4+ T cells, expanded with dendritic cells presenting a single autoantigenic peptide, suppress autoimmune diabetes. The Journal of experimental medicine 2004; 199:1467-77.

10. Bluestone J A, Tang Q. Therapeutic vaccination using CD4+CD25+ antigen-specific regulatory T cells. Proceedings of the National Academy of Sciences of the United States of America 2004; 101 Suppl 2:14622-6.

11. Luo X, Herold K C, Miller S D. Immunotherapy of type 1 diabetes: where are we and where should we be going? Immunity 2010; 32:488-99.

12. Nakayama M, Abiru N, Moriyama H, et al. Prime role for an insulin epitope in the development of type 1 diabetes in NOD mice. Nature 2005; 435:220-3.

13. Kendal A R, Waldmann H. Infectious tolerance: therapeutic potential. Current opinion in immunology 2010; 22:560-5.

14. McCarthy D P, Bryant J, Galvin J P, Miller S D, Luo X. Tempering allorecognition to induce transplant tolerance with chemically modified apoptotic donor cells. American journal of transplantation: official journal of the American Society of Transplantation and the American Society of Transplant Surgeons 2015; 15:1475-83.

15. Miller S D, Turley D M, Podojil J R. Antigen-specific tolerance strategies for the prevention and treatment of autoimmune disease. Nature reviews Immunology 2007; 7:665-77.

16. Fife BT, Guleria I, Gubbels Bupp M, et al. Insulin-induced remission in new-onset NOD mice is maintained by the PD-1-PD-L1 pathway. The Journal of experimental medicine 2006; 203:2737-47.

17. Turley D M, Miller SD. Peripheral tolerance induction using ethylenecarbodiimide-fixed APCs uses both direct and indirect mechanisms of antigen presentation for prevention of experimental autoimmune encephalomyelitis. J Immunol 2007; 178:2212-20.

18. Chen G, Sun H, Yang H, et al. The role of anti-non-Gal antibodies in the development of acute humoral xenograft rejection of hDAF transgenic porcine kidneys in baboons receiving anti-Gal antibody neutralization therapy. Transplantation 2006; 81:273-83.

19. Smarr C B, Hsu C L, Byrne A J, Miller S D, Bryce P J. Antigen-fixed leukocytes tolerize Th2 responses in mouse models of allergy. J Immunol 2011; 187:5090-8.

20. Luo X, Pothoven K L, McCarthy D, et al. ECDI-fixed allogeneic splenocytes induce donor-specific tolerance for long-term survival of islet transplants via two distinct mechanisms. Proceedings of the National Academy of Sciences of the United States of America 2008; 105:14527-32.

21. Wang S, Tasch J, Kheradmand T, et al. Transient B-cell depletion combined with apoptotic donor splenocytes induces xeno-specific T- and B-cell tolerance to islet xenografts. Diabetes 2013; 62:3143-50.

22. Lutterotti A, Yousef S, Sputtek A, et al. Antigen-specific tolerance by autologous myelin Peptide-coupled cells: a phase 1 trial in multiple sclerosis. Science translational medicine 2013; 5: 188ra75.

23. Kheradmand T, Wang S, Bryant J, et al. Ethylenecarbodiimide-fixed donor splenocyte infusions differentially target direct and indirect pathways of allorecognition for induction of transplant tolerance. J Immunol 2012; 189:804-12.

24. Lei J, Kim J I, Shi S, et al. Pilot Study Evaluating Regulatory T Cell-Promoting Immunosuppression and Nonimmunogenic Donor Antigen Delivery in a Nonhuman Primate Islet Allotransplantation Model. American journal of transplantation: official journal of the American Society of Transplantation and the American Society of Transplant Surgeons 2015; 15:2739-49.

25. Bryant J, Hlavaty K A, Zhang X, et al. Nanoparticle delivery of donor antigens for transplant tolerance in allogeneic islet transplantation. Biomaterials 2014; 35:8887-94.

26. Getts D R, Martin A J, McCarthy D P, et al. Microparticles bearing encephalitogenic peptides induce T-cell tolerance and ameliorate experimental autoimmune encephalomyelitis. Nature biotechnology 2013; 31:565.

27. Getts D R, Terry R L, Getts M T, et al. Therapeutic inflammatory monocyte modulation using immune- modifying microparticles. Science translational medicine 2014; 6:219ra7.

28. Getts D R, McCarthy D P, Miller S D. Exploiting apoptosis for therapeutic tolerance induction. J Immunol 2013; 191:5341-6.

29. Chen G, Kheradmand T, Bryant J, et al. Intragraft CD11b(+) ID0(+) cells mediate cardiac allograft tolerance by ECDI-fixed donor splenocyte infusions. American journal of transplantation : official journal of the American Society of Transplantation and the American Society of Transplant Surgeons 2012; 12:2920- 9.

30. Lemke G, Rothlin C V. Immunobiology of the TAM receptors. Nature reviews Immunology 2008; 8:327-36.

31. Scott R S, McMahon E J, Pop S M, et al. Phagocytosis and clearance of apoptotic cells is mediated by MER. Nature 2001; 411:207-11.

32. Lemke G, Burstyn-Cohen T. TAM receptors and the clearance of apoptotic cells. Annals of the New York Academy of Sciences 2010; 1209:23-9.

33. Lu Q, Lemke G. Homeostatic regulation of the immune system by receptor tyrosine kinases of the Tyro 3 family. Science 2001; 293:306-11.

34. Rothlin C V, Ghosh S, Zuniga E I, Oldstone M B, Lemke G. TAM receptors are pleiotropic inhibitors of the innate immune response. Cell 2007; 131:1124-36.

35. Zagorska A, Traves P G, Lew E D, Dransfield I, Lemke G. Diversification of TAM receptor tyrosine kinase function. Nature immunology 2014; 15:9208.

36. Bhattacharyya S, Zagorska A, Lew E D, et al. Enveloped viruses disable innate immune responses in dendritic cells by direct activation of TAM receptors. Cell host & microbe 2013; 14:136-47.

37. Conde P, Rodriguez M, van der Touw W, et al. DC-SIGN(+) Macrophages Control the Induction of Transplantation Tolerance. Immunity 2015; 42:1143-58.

38. Meyer S, van Liempt E, Imberty A, et al. DC-SIGN mediates binding of dendritic cells to authentic pseudo- LewisY glycolipids of Schistosoma mansoni cercariae, the first parasite-specific ligand of DC-SIGN. The Journal of biological chemistry 2005; 280:37349-59.

39. Srivastava L, Tundup S, Choi B S, Norberg T, Harn D. Immunomodulatory glycanlacto-N-fucopentaose III requires clathrin-mediated endocytosis to induce alternative activation of antigen-presenting cells. Infection and immunity 2014; 82:1891-903.

40. Tundup S, Srivastava L, Norberg T, Watford W, Harn D. A Neoglycoconjugate Containing the Human Milk Sugar LNFPIII Drives Anti-Inflammatory Activation of Antigen Presenting Cells in a CD14 Dependent Pathway. PloS one 2015; 10:e0137495.

41. Dutta P, Hullett D A, Roenneburg D A, et al. Lacto-N-fucopentaose III, a pentasaccharide, prolongs heart transplant survival. Transplantation 2010; 90:1071-8.

42. Schvartz D, Brunner Y, Coute Y, Foti M, Wollheim C B, Sanchez J C. Improved characterization of the insulin secretory granule proteomes. Journal of proteomics 2012; 75:4620-31.

43. Brunner Y, Coute Y, Iezzi M, et al. Proteomics analysis of insulin secretory granules. Molecular & cellular proteomics : MCP 2007; 6:1007-17.

44. Hickey A J, Bradley J W, Skea G L, et al. Proteins associated with immunopurified granules from a model pancreatic islet beta-cell system: proteomic snapshot of an endocrine secretory granule. Journal of proteome research 2009; 8:178-86.

45. Stadinski B D, Delong T, Reisdorph N, et al. Chromogranin A is an autoantigen in type 1 diabetes. Nature immunology 2010; 11:225-31.

46. Grubin C E, Kovats S, deRoos P, Rudensky A Y. Deficient positive selection of CD4 T cells in mice displaying altered repertoires of MHC class II-bound self-peptides. Immunity 1997; 7:197-208.

47. Brennan T V, bang V, Garrod K R, et al. A new T-cell receptor transgenic model of the CD4+ direct pathway: level of priming determines acute versus chronic rejection. Transplantation 2008; 85:247-55.

48. Bryant J, Lerret N M, Wang J J, et al. Preemptive Donor Apoptotic Cell Infusions Induce IFN-gamma-Producing Myeloid-Derived Suppressor Cells for Cardiac Allograft Protection. J Immunol 2014; 192:6092- 101.

49. Hlavaty K A, McCarthy D P, Saito E, Yap W T, Miller S D, Shea L D. Tolerance induction using nanoparticles bearing HY peptides in bone marrow transplantation. Biomaterials 2015; 76:1-10.

50. Hoshi R A, Van Lith R, Jen M C, Allen J B, Lapidos K A, Ameer G. The blood and vascular cell compatibility of heparin-modified ePTFE vascular grafts. Biomaterials 2013; 34:30-41.

51. Best M D. Click chemistry and bioorthogonal reactions: unprecedented selectivity in the labeling of biological molecules. Biochemistry 2009; 48:6571-84.

52. Shang J, Piskarev V E, Xia M, et al. Identifying human milk glycans that inhibit norovirus binding using surface plasmon resonance. Glycobiology 2013; 23:1491-8.

53. Yang J, van Lith R, Baler K, Hoshi R A, Ameer G A. A thermoresponsive biodegradable polymer with intrinsic antioxidant properties. Biomacromolecules 2014; 15:3942-52.

54. Hunter Z, McCarthy D P, Yap W T, et al. A biodegradable nanoparticle platform for the induction of antigen-specific immune tolerance for treatment of autoimmune disease. ACS nano 2014; 8:2148-60.

55. Whittlesey K J, Shea L D. Delivery systems for small molecule drugs, proteins, and DNA: the neuroscience/biomaterial interface. Experimental neurology 2004; 190:1-16.

56. Yap W T, Song W K, Chauhan N, et al. Quantification of particle-conjugated or particleencapsulated peptides on interfering reagent backgrounds. BioTechniques in press.

57. Shin S, Tuinstra H M, Salvay D M, Shea L D. Phosphatidylserine immobilization of lentivirus for localized gene transfer. Biomaterials 2010; 31:4353-9.

58. Luo X, Yang H, Kim I S, et al. Systemic transforming growth factor-betal gene therapy induces Foxp3+ regulatory cells, restores self-tolerance, and facilitates regeneration of beta cell function in overtly diabetic nonobese diabetic mice. Transplantation 2005; 79:1091-6.

59. Prasad S, Kohm A P, McMahon J S, Luo X, Miller S D. Pathogenesis of NOD diabetes is initiated by reactivity to the insulin B chain 9-23 epitope and involves functional epitope spreading. Journal of autoimmunity 2012; 39:347-53.

60. Ishihara H, Asano T, Tsukuda K, et al. Pancreatic beta cell line MING exhibits characteristics of glucose metabolism and glucose-stimulated insulin secretion similar to those of normal islets. Diabetologia 1993; 36:1139-45.

61. Krishnamurthy B, Mariana L, Gellert S A, et al. Autoimmunity to both proinsulin and IGRP is required for diabetes in nonobese diabetic 8.3 TCR transgenic mice. J Immunol 2008; 180:4458-64.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. Carbohydrate-modified particles, the particles comprising a biodegradable polymeric base material having an effective average diameter of 0.01-500 pm and a carbohydrate moiety that is an immune modulator covalently attached at the surface of the particles.

2. The particles of claim 1, wherein the carbohydrate moiety is selected from a group consisting of Heparin disaccharide I-A, Heparin disaccharide II-A, Heparin disaccharide III-A, Heparin disaccharide IV-A, Heparin disaccharide IV-S, Heparin unsaturated disaccharide I-H, Heparin unsaturated disaccharide II-H, Heparin unsaturated disaccharide II-H, Heparin unsaturated disaccharide I-P, Chondroitin disaccharide Adi-OS, Chondroitin disaccharide Adi-45, Chondroitin disaccharide Adi-65, Chondroitin disaccharide ADi-diSB, Chondroitin disaccharide ADi-diSE, Chondroitin disaccharide ADi-triS, Chondroitin disaccharide ADi-UA2S, Neocarradecaose-41 ,3,5,7,9-penta-0-sulphate, neocarrahexadecaose-41,3,5,7,9,11,13,15-octa-0-sulfate, GalNAcj31-4Gal (receptor for pili of Pseudomonas aeruginosa), Blood group B type 2 linear trisaccharide, P1 Antigen, Tn Antigen, Sialyl-Lewis A, Sialyl-Lewis X, Sialyl-Lewis X j3-methyl glycoside, Sulfo-Lewis A, Sulfo-Lewis X, al-2-Mannobiose, al-3-Mannobiose, al-6-Mannobiose, Mannotetraose, al-3, al-3, al-6-Mannopentose, 131-2-N-Acetylglucosamine-mannose, LS-Tetrasaccharide a (LSTa), LS-tetrasaccharide c (LSTc), a-D-N-Acetylgalactosaminyl 1-3 galactose, a-D-N-Acetylgalactosaminyl 1-3 galactose 131-4 glucose, D-Galactose-4-O-sulfate, Glycyl-lactose (Lac-gly), Glycyl-lacto-N-tetraose (LNT-gly), 2′-Fucosyllactose, Lacto-N-neotetraose (LNnT), Lacto-N-tetraose (LNT), Lacto-N-difucohexaose I (LNDFH I), Lacto-N-difucohexaose II (LNDFHII), Lacto-N-neohexaose (LNnH), 3′-Sialyllactose (3′-SL), 6′-Sialyllactose (6′-SL), 3′-Sialyl-N-acetyllactosamine, 6′-Sialyl-N-acetyllactosamine (6′-SLN), 3-Fucosyllactose (3FL), Fucoidan, 4-13-Galactobiose, 1-3 Galactodiosyl 13-methyl glycosie, al-3, 131-4, al-3 Galactotetraose, 13-Galactosyl 1-3 N-acetyl galactosamine methyl glycoside, 131-3 Gal-N-acetyl galactosaminyl-131-4 Gal-131-4-Glc, 131-6 Galactobiose, Globotriose, 13-D-N-Acetylglactosaminyl 1-3 galactose (terminal disaccharide of globotriose), 1-Deoxynojirimyncin (DNJ), D-Fucose, L-Fucose, D-Talose, Calystegine A3, Calystegine B3, N-methyl cis-4-hydroxymehtyl-L-proline, 2,5-dideoxy-2,5-imino-D-mannitol, Castanospermine, 6-epi-Castanospermine, and combinations thereof.

3. The particles of claim 1, wherein the polymeric base material comprises a co-polymer of polylactic acid (PLA) and polyglycolic acid (PGA) (i. e., PLGA).

4. The particles of claim 1, wherein the carbohydrate moiety is covalently attached to the surface of the particles via a linker.

5. The particles of claim 4, wherein the linker comprises: (1) an electrophile that reacts with a free hydroxyl group of the carbohydrate moiety; and (2) a nucleophile that reacts with a free carboxyl group of the polymeric base material.

6. The particles of claim 5, wherein the carbohydrate moiety is covalently attached to the surface of the particles via carbodiimide crosslinking

7. The particles of claim 1, further comprising an additional immunomodulator other than the carbohydrate moiety.

8. The particles of claim 1, wherein the immune modulator induces desensitization or tolerance and/or the immune modulator induces an anti-inflammatory response.

9. The particles of claim 8, wherein the additional immunomodulator is an antigen associated with an autoimmune disease or disorder.

10. The particles of claim 9, wherein the antigen is an antigen derived from insulin.

11. A pharmaceutical composition comprising the particles of claim 1 together with a suitable carrier, excipient, or diluent.

12. A method for treating a disease or disorder in a subject in need thereof, the method comprising administering the composition of claim 11 to the subject.

13. The method of claim 12, wherein the subject has or is at risk for developing an immune disease or disorder.

14. The method of claim 13, wherein the immune disease or disorder is an allergic reaction and the method induces tolerance in the subject.

15. The method of claim 13, wherein the immune disease or disorder is an autoimmune disease or disorder.

16. The method of claim 15, wherein the immune disease or disorder is diabetes mellitus type 1.

17. A method for preparing the particles of claim 1, the method comprising one or more of the following steps:
(a) screening a library of carbohydrate moieties for immune modulator activity by contacting the library with an immune cell and measuring the effect of the library on stimulating the immune cell;
(b)selecting a carbohydrate moiety based on its effect on stimulating the immune cell; and
(c) attaching the carbohydrate moiety to particles formed from a polymeric base material.

18. The method of claim 17, wherein measuring the effect of the library on stimulating the immune cell comprising measuring cytokine production.

19. The method of claim 18, wherein measuring cytokine production comprises measuring IL-10 production over baseline and measuring IL-6 production over baseline, and selecting a carbohydrate moiety based on its effect on stimulating the immune cell comprises selected a carbohydrate moiety that increases IL-10 secretion over baseline while not changing IL-6 secretion or while decreasing IL-6 secretion.

20. The method of claim 17, wherein attaching the carbohydrate moiety is attached covalently to particles formed from a polymeric base material.


SEQUENCE LISTING (9 sequences)

SEQ ID NO: 1 - protein, 15 aa, Homo sapiens
GAGDLQTLALEVAQQ

SEQ ID NO: 2 - protein, 15 aa, Homo sapiens
GGGPGAGDLETLALE

SEQ ID NO: 3 - protein, 15 aa, Homo sapiens
GGGPGAGDLQTLALE

SEQ ID NO: 4 - protein, 15 aa, Homo sapiens
ELELGGGPGAGDLET

SEQ ID NO: 5 - protein, 15 aa, Homo sapiens
QLELGGGPGAGDLET

SEQ ID NO: 6 - protein, 15 aa, Homo sapiens
ELELGGGPGAGDLQT

SEQ ID NO: 7 - protein, 15 aa, Homo sapiens
QLELGGGPGAGDLQT

SEQ ID NO: 8 - protein, 15 aa, Homo sapiens
PEVAELELGGGPGAG

SEQ ID NO: 9 - protein, 86 aa, Homo sapiens
FVKEHLCGSHLVEALYLVCGERGFFYTPMSRREVEDPQVAELELGGGPGAGDLETLALEVAQEKRGIVDECCTSICSLYELENYCN